(19) United States
(12) Patent Application Publication
(10) Pub. No.: US 2023/0331670 A1
(43) Pub. Date: Oct. 19, 2023

(54) GLYCEROL COMPOUNDS AND METHODS OF USE

(71) Applicant: Enzychem Lifesciences Corporation, Seoul Daejeon (KR)

(72) Inventors: Kaapjoo Park, Seoul Daejeon (KR); Sun Young Yoon, Seoul Daejeon (KR); Dong Gyun Shin, Seoul Daejeon (KR)

(21) Appl. No.: 18/027,080

(22) PCT Filed: Sep. 17, 2021
(86) PCT No.: PCT/IB2021/058516

§ 371 Date: Mar. 17, 2023

Related U.S. Application Data
(60) Provisional application No. 63/080,007, filed on Sep. 17, 2020.

(51) Int. Cl. C07D 207/27; C07C 69/732; C07C 43/15

(52) U.S. Cl. CPC C07D 207/27; C07C 69/732; C07C 43/15

(57) ABSTRACT

Provided herein, inter alia, are compounds of fatty acid glycerol derivatives and compositions including the same.

### CROSS-REFERENCES TO RELATED APPLICATIONS

This application claims the benefit of U.S. Provisional Application No. 63/080,007, filed Sep. 17, 2020, which is incorporated herein by reference in its entirety and for all purposes.

### FIELD

In one aspect, new glycerol compounds and compositions are provided. The compositions include pharmaceutical compositions and related methods of treatment.

### BACKGROUND

PLAG (1-palmitoyl-3-linoleoyl-3-acetylglycerol) has been demonstrated for treating various diseases such as acute radiation syndrome (ARS) and cancer. See WO2019/106632.

It would be desirable to have additional compounds for treatment of such disorders.

### SUMMARY

We now provide new compounds and compositions that can be useful for treatment and prevention of disease.

More particularly, in one aspect, compounds are provided that comprise a structure of the following Formula (I):

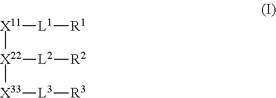

X¹¹ is —CR^{1a}R^{1b}—, C(O)— or —NR^{1c}—.

X²² is —CR^{2a}— or —N—.

X³³ is —CR^{3a}R^{3b}—, —C(O)— or —NR^{3c}—.

L¹ is a bond, —C(O)—, —C(O)O—, —OC(O)—, —C(O)S—, SC(O)—, —NR¹¹C(O)—, —C(O)NR¹¹—, —NR¹¹C(O)NR¹²—, —NR¹¹—, —O—, —S—, —S(O)₂—, —NR¹¹S(O)₂—, —S(O)₂NR¹¹—, —NR¹¹C(O)O—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

L² is a bond, —C(O)—, —C(O)O—, —OC(O)—, —C(O)S—, SC(O)—, —NR¹³C(O)—, —C(O)NR¹⁴—, —NR¹³C(O)NR¹⁴—, —NR¹³—, —O—, —S—, —S(O)₂—, —NR¹³S(O)₂—, —S(O)₂NR¹³—, —NR¹³C(O)O—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

L³ is a bond, —C(O)—, —C(O)O—, —OC(O)—, —C(O)S—, SC(O)—, —NR¹⁵C(O)—, —C(O)NR¹⁵—, —NR¹⁵C(O)NR¹⁶—, —NR¹⁵—, —O—, —S—, —S(O)₂—, —NR¹⁵S(O)₂—, —S(O)₂NR¹⁵—, —NR¹⁵C(O)O—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

R¹ is —CX¹₃, —CHX¹₂, —CH₂X¹, —OCX¹₃, —OCH₂X¹, —OCHX¹₂, —N₃, —CN, —SO₂R^{1D}, —SO₂NR^{1A}R^{1B}, —NHC(O)NR^{1A}R^{1B}, —NO₂, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R² is hydrogen, halogen, —CX²₃, —CHX²₂, —CH₂X², —OCX²₃, —OCH₂X², —OCHX²₂, —N₃, —CN, —SO₂R^{2D}, —SO₂NR^{2A}R^{2B}, —NHC(O)NR^{2A}R^{2B}, —NO₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or R^{2a} and R² together with atoms attached thereto are optionally joined to form a substituted or unsubstituted cycloalkyl, or substituted or unsubstituted heterocycloalkyl.

R³ is hydrogen, halogen, —CX³₃, —CHX³₂, —CH₂X³, —OCX³₃, —OCH₂X³, —OCHX³₂, —N₃, —CN, —SO₂R^{3D}, —SO₂NR^{3A}R^{3B}, —NHC(O)NR^{3A}R^{3B}, —NO₂, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3c}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

wherein R¹¹, R¹², R¹³, R¹⁴, R¹⁵, R¹⁶, R^{1a}, R^{1b}, R^{1c}, R^{2a}, R^{3a}, R^{3b}, R^{3c}, R^{1A}, R^{1B}, R^{1C}, R^{1D}, R^{2A}, R^{2B}, R^{2C}, R^{2D}, R^{3A}, R^{3B}, R^{3C}, and R^{3D} are independently hydrogen, —CX₃, —CHX₂, —CH₂X, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

X, X¹, X², and X³ are independently —F, —Cl, —Br, or —I.

In some embodiments, preferred compounds may have a structure of the following Formula (II):

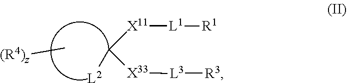

wherein L² is a bond, —C(O)—, —OC(O)—, —SC(O)—, —C(O)O—, —C(O)S—, —NHC(O)—, —C(O)NH—, —NHC(O)NH—, —NH—, —NCH₃—, —O—, —S—, —S(O)₂—, —NHS(O)₂—, —S(O)₂NH—, —NHC(O)O—, —OC(O)NH, substituted or unsubstituted C₁-C₃ alkylene, or substituted or unsubstituted 2 to 3 membered heteroalkylene;

z is an integer from 0 to 8;

R⁴ is halogen, —CX⁴₃, —CHX⁴₂, —CH₂X⁴, —OCX⁴₃, —OCH₂X⁴, —OCHX⁴₂, —N₃, —CN, —SO₂R^{4D}, —SO₂NR^{4A}R^{4B}, —NHC(O)NR^{4A}R^{4B}, —NO₂, —NR^{4A}R^{4B}, —C(O)R^{4C}, —C(O)—OR^{4C}, —C(O)NR^{4A}R^{4B}, —OR^{4D}, —NR^{4A}SO₂R^{4D}, —NR^{4A}C(O)R^{4C}, —NR^{4A}C(O)OR^{4C}, —NR^{4A}OR^{4C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R^{4A}, R^{4B}, R^{4C}, and R^{4D} are independently hydrogen, —CX₃, —CHX₂, —CH₂X, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

X⁴ is independently —F, Cl, —Br, or —I.

In some embodiments, preferred compound may comprise a structure of the compound has the structure of either the following Formulae (III-a) or (III-b):

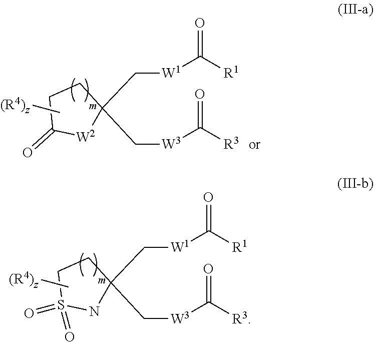

In formula (III-a), each W¹, W², and W³ is independently —NH—, —O—, or —S—; and m is an integer from 0 to 4

In some embodiments, preferred compounds may comprise a structure of the following Formula (IV):

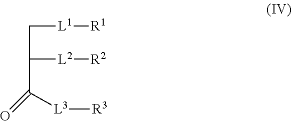

wherein R¹, R² and R³ are as described above.

In formula (IV), each L¹, L², and L³ is independently a bond, —OC(O)—, —SC(O)—, or —NHC(O)—.

In some embodiments, the compound has the structure of the following Formula (V-a) or (V-b):

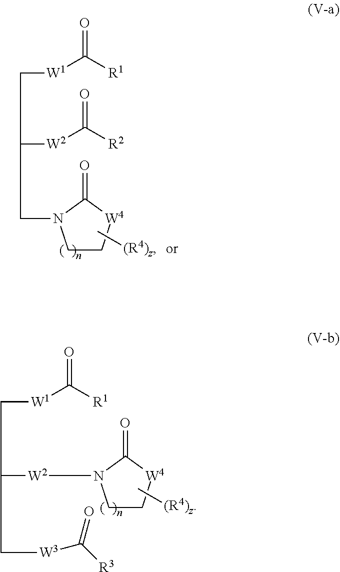

In formula (V-a) or (V-b), each W¹ and W² is independently a bond, —NH—, —O—, or —S—. W⁴ is —NR¹⁶—, —CH₂—, —O—.

n is an integer from 0 to 4.

z is an integer from 0 to 8.

R⁴ is halogen, —CX⁴₃, —CHX⁴₂, —CH₂X⁴, —OCX⁴₃, —OCH₂X⁴, —OCHX⁴₂, —N₃, —CN, —SO₂R^{4D}, —SO₂NR^{4A}R^{4B}, —NHC(O)NR^{4A}R^{4B}, —NO₂, —NR^{4A}R^{4B}, —C(O)R^{4C}, —C(O)—OR^{4C}, —C(O)NR^{4A}R^{4B}, —OR^{4D}, —NR^{4A}SO₂R^{4D}, —NR^{4A}C(O)R^{4C}, —NR^{4A}C(O)OR^{4C}, —NR^{4A}OR^{4C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R^{4A}, R^{4B}, R^{4C}, and R^{4D} are independently hydrogen, —CX₃, —CHX₂, —CH₂X, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

X⁴ is independently —F, Cl, —Br, or —I.

In an aspect, we also provide a pharmaceutical composition including the compound as described herein and optionally one or more pharmaceutically acceptable excipients.

In an aspect, further provided is a method of treating a patient suffering from cancer, including administering a compound as described herein to a patient that is suffering from cancer, including a solid tumor.

In a yet further aspect, methods are also provided to treat a patient suffering from or susceptible to acute radiation syndrome, including administering a compound as described herein to a patient that is suffering from or susceptible to acute radiation syndrome.

Methods are also provided to treat a subject that has been exposed to ionizing radiation (particularly adverse exposure such as unintended and/or non-therapeutic exposure, and/or exposure to excessive ionizing radiation, including gamma radiation) which include administering to the subject an effective amount of a compound disclosed herein.

In a yet further aspect, methods are also provided to treat a patient suffering from or susceptible to inflammation, including administering a compound as described herein to a patient that is suffering from or susceptible to acute lung injury or mucositis, including oral mucositis (e.g., oral ulceration) or gastrointestinal mucositis.

In another aspect, provided is a kit for treating a condition as disclosed herein. Preferred kits may include a therapeutically effective amount of a compound as disclosed herein and instructions including written instructions for treatment of a disease or disorder of exposure to ionizing radiation acute lung injury or mucositis.

Other aspects are disclosed infra.

### DETAILED DESCRIPTION

### I. Definitions

The abbreviations used herein have their conventional meaning within the chemical and biological arts. The chemical structures and formulae set forth herein are constructed according to the standard rules of chemical valency known in the chemical arts.

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., O, N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—S—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P).

A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. In embodiments, bridged monocyclic rings contain a monocyclic cycloalkyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. In embodiments, fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkyl is attached to the parent molecular moiety through any carbon atom contained within the monocyclic cycloalkyl ring. In embodiments, cycloalkyl groups are optionally substituted with one or two groups which are independently oxo or thia. In embodiments, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused bicyclic cycloalkyl is optionally substituted by one or two groups which are independently oxo or thia. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkyl is attached to the parent molecular moiety through any carbon atom contained within the base ring. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic cycloalkyl groups include, but are not limited to tetradecahydrophenanthrenyl, perhydrophenothiazin-1-yl, and perhydrophenoxazin-1-yl.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. In embodiments, monocyclic cycloalkenyl ring systems are cyclic hydrocarbon groups containing from 3 to 8 carbon atoms, where such groups are unsaturated (i.e., containing at least one annular carbon carbon double bond), but not aromatic. Examples of monocyclic cycloalkenyl ring systems include cyclopentenyl and cyclohexenyl. In embodiments, bicyclic cycloalkenyl rings are bridged monocyclic rings or a fused bicyclic rings. In embodiments, bridged monocyclic rings contain a monocyclic cycloalkenyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic cycloalkenyls include, but are not limited to, norbornenyl and bicyclo[2.2.2]oct 2 enyl. In embodiments, fused bicyclic cycloalkenyl ring systems contain a monocyclic cycloalkenyl ring fused to either a phenyl, a monocyclic cycloalkyl, a monocyclic cycloalkenyl, a monocyclic heterocyclyl, or a monocyclic heteroaryl. In embodiments, the bridged or fused bicyclic cycloalkenyl is attached to the parent molecular moiety through any carbon atom contained within the monocyclic cycloalkenyl ring. In embodiments, cycloalkenyl groups are optionally substituted with one or two groups which are independently oxo or thia. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In embodiments, the multicyclic cycloalkenyl is attached to the parent molecular moiety through any carbon atom contained within the base ring. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a monocyclic heteroaryl, a monocyclic cycloalkyl, a monocyclic cycloalkenyl, and a monocyclic heterocyclyl.

In embodiments, a heterocycloalkyl is a heterocyclyl. The term “heterocyclyl” as used herein, means a monocyclic, bicyclic, or multicyclic heterocycle. The heterocyclyl monocyclic heterocycle is a 3, 4, 5, 6 or 7 membered ring containing at least one heteroatom independently selected from the group consisting of O, N, and S where the ring is saturated or unsaturated, but not aromatic. The 3 or 4 membered ring contains 1 heteroatom selected from the group consisting of O, N and S. The 5 membered ring can contain zero or one double bond and one, two or three heteroatoms selected from the group consisting of O, N and S. The 6 or 7 membered ring contains zero, one or two double bonds and one, two or three heteroatoms selected from the group consisting of O, N and S. The heterocyclyl monocyclic heterocycle is connected to the parent molecular moiety through any carbon atom or any nitrogen atom contained within the heterocyclyl monocyclic heterocycle. Representative examples of heterocyclyl monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The heterocyclyl bicyclic heterocycle is a monocyclic heterocycle fused to either a phenyl, a monocyclic cycloalkyl, a monocyclic cycloalkenyl, a monocyclic heterocycle, or a monocyclic heteroaryl. The heterocyclyl bicyclic heterocycle is connected to the parent molecular moiety through any carbon atom or any nitrogen atom contained within the monocyclic heterocycle portion of the bicyclic ring system. Representative examples of bicyclic heterocyclyls include, but are not limited to, 2,3-dihydrobenzofuran-2-yl, 2,3-dihydrobenzofuran-3-yl, indolin-1-yl, indolin-2-yl, indolin-3-yl, 2,3-dihydrobenzothien-2-yl, decahydroquinolinyl, decahydroisoquinolinyl, octahydro-1H-indolyl, and octahydrobenzofuranyl. In embodiments, heterocyclyl groups are optionally substituted with one or two groups which are independently oxo or thia. In certain embodiments, the bicyclic heterocyclyl is a 5 or 6 membered monocyclic heterocyclyl ring fused to a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the bicyclic heterocyclyl is optionally substituted by one or two groups which are independently oxo or thia. Multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. The multicyclic heterocyclyl is attached to the parent molecular moiety through any carbon atom or nitrogen atom contained within the base ring. In embodiments, multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic heterocyclyl groups include, but are not limited to 10H-phenothiazin-10-yl, 9,10-dihydroacridin-9-yl, 9,10-dihydroacridin-10-yl, 10H-phenoxazin-10-yl, 10,11-dihydro-5H-dibenzo[b,f]azepin-5-yl, 1,2,3,4-tetrahydropyrido[4,3-g]isoquinolin-2-yl, 12H-benzo[b]phenoxazin-12-yl, and dodecahydro-1H-carbazol-9-yl.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substitutents described herein.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g. substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the remainder of a molecule or chemical formula.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds disclosed herein may exist in tautomeric forms, all such tautomeric forms of the compounds being within the scope of the invention.

The terms “a” or “an,” as used in herein means one or more. For example, the singular forms “a,” “an” and “the” are intended to include the plural forms as well, unless the context clearly indicates otherwise. It will be further understood that the terms “comprise”, “include”, “have”, etc. when used in this specification, specify the presence of stated features, regions, integers, steps, processes, operations, elements and/or components but do not preclude the presence or addition of one or more other features, regions, integers, steps, processes, operations, elements, components, and/or combinations thereof.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. The term “treating” and conjugations thereof, may include prevention of an injury, pathology, condition, or disease. In embodiments, treating is preventing. In embodiments, treating does not include preventing.

The term “prevent” refers to a decrease in the occurrence of disease symptoms in a patient. As indicated above, the prevention may be complete (e.g., no detectable symptoms) or partial, such that fewer symptoms are observed than would likely occur absent treatment.

The term “modulate” is used in accordance with its plain ordinary meaning and refers to the act of changing or varying one or more properties. “Modulation” refers to the process of changing or varying one or more properties. For example, a modulator of a disease decreases a symptom, cause, or characteristic of the targeted disease such as ARS and its subsyndromes.

“Patient,” “subject,” “patient in need thereof,” and “subject in need thereof” are herein used interchangeably and refer to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient or subject is human.

An “effective amount” is an amount sufficient for a compound to accomplish a stated purpose relative to the absence of the compound (e.g. achieve the effect for which it is administered, treat a disease, reduce enzyme activity, increase enzyme activity, reduce a catabolic enzyme activity, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

A therapeutically effective amount of the compounds as described herein can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The term “therapeutically effective amount” or “effective amount” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound being employed. The dose administered to a patient, in the context of the present invention should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal) compatible with the preparation. Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

The compositions disclosed herein can be delivered by transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions disclosed herein can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In another embodiment, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions can also be delivered as nanoparticles.

Pharmaceutical compositions may include compositions wherein the compound described herein is contained in a therapeutically effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., modulating the activity of a target molecule, and/or reducing, eliminating, or slowing the progression of disease symptoms.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated, kind of concurrent treatment, complications from the disease being treated or other health-related problems. Other therapeutic regimens or agents can be used in conjunction with the methods and compounds of Applicants' invention. Adjustment and manipulation of established dosages (e.g., frequency and duration) are well within the ability of those skilled in the art.

“Disease”, “disorder” or “condition” refer to a state of being or health status of a patient or subject capable of being treated with the compounds or methods provided herein.

### II. Compounds

In one aspect, provided is a compound having a structure of:

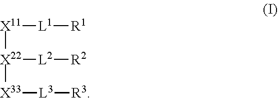

X¹¹ is —CR^{1a}R^{1b}—, C(O)— or —NR^{1c}—.

X²² is —CR^{2a}— or —N—.

X³³ is —CR^{3a}R^{3b}—, —C(O)— or —NR^{3c}—.

L¹ is a bond, —C(O)—, —C(O)O—, —OC(O)—, —C(O)S—, SC(O)—, —NR¹¹C(O)—, —C(O)NR¹¹—, —NR¹¹C(O)NR¹²—, —NR¹¹—, —O—, —S—, —S(O)₂—, —NR¹¹S(O)₂—, —S(O)₂NR¹¹—, —NR¹¹C(O)O—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

L² is a bond, —C(O)—, —C(O)O—, —OC(O)—, —C(O)S—, SC(O)—, —NR¹³C(O)—, —C(O)NR¹⁴—, —NR¹³C(O)NR¹⁴—, —NR¹³—, —O—, —S—, —S(O)₂—, —NR¹³S(O)₂—, —S(O)₂NR¹³—, —NR¹³C(O)O—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

L³ is a bond, —C(O)—, —C(O)O—, —OC(O)—, —C(O)S—, SC(O)—, —NR¹⁵C(O)—, —C(O)NR¹⁵—, —NR¹⁵C(O)NR¹⁶—, —NR¹⁵—, —O—, —S—, —S(O)₂—, —NR¹⁵S(O)₂—, —S(O)₂NR¹⁵—, —NR¹⁵C(O)O—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

R¹ is —CX¹₃, —CHX¹₂, —CH₂X¹, —OCX¹₃, —OCH₂X¹, —OCHX¹₂, —N₃, —CN, —SO₂R^{1D}, —SO₂NR^{1A}R^{1B}, —NHC(O)NR^{1A}R^{1B}, —NO₂, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R² is hydrogen, halogen, —CX²₃, —CHX²₂, —CH₂X², —OCX²₃, —OCH₂X², —OCHX²₂, —N₃, —CN, —SO₂R^{2D}, —SO₂NR^{2A}R^{2B}, —NHC(O)NR^{2A}R^{2B}, —NO₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)O R^{2C}, —NR^{2A}OR^{2C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or R^{2a} and R² together with atoms attached thereto are optionally joined to form a substituted or unsubstituted cycloalkyl, or substituted or unsubstituted heterocycloalkyl.

R³ is hydrogen, halogen, —CX³₃, —CHX³₂, —CH₂X³, —OCX³₃, —OCH₂X³, —OCHX³₂, —N₃, —CN, —SO₂R^{3D}, —SO₂NR^{3A}R^{3B}, —NHC(O)NR^{3A}R^{3B}, —NO₂, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R¹¹1, R¹², R¹³, R¹⁴, R¹⁵, R¹⁶, R^{1a}, R^{1b}, R^{1c}, R^{2a}, R^{3a}, R^{3b}, R^{3c}, R^{1A}, R^{1B}, R^{1C}, R^{1D}, R^{2A}, R^{2B}, R^{2C}, R^{2D}, R^{3A}, R^{3B}, R^{3C}, and R^{3D} are independently hydrogen, —CX₃, —CHX₂, —CH₂X, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

X, X¹, X², and X³ are independently —F, —Cl, —Br, or —I.

In certain embodiments, the compounds does not include any compounds provided in PCT/KR2019/002757 (published as WO2019/177314 A1 on Sep. 19, 2020).

In certain embodiments, the compounds does not include any compounds provided in PCT/KR2019/003437 (published as WO2019/190137 A1 on Oct. 3, 2019).

In certain embodiments, the compounds does not include any compounds provided in PCT/KR2019/004789 (published as WO2019/208980 A1 on Oct. 31, 2019).

In certain embodiments, when X¹¹, X²², X³³ are —CH₂—; L¹, L², and L³ are —OC(O)—; R¹ is C₁₅ unsubstituted alkyl; and R³ is methyl, then R² is not

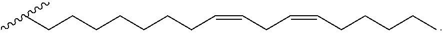 [SMILES: *CCCCCCC/C=C\C/C=C\CCCCC]

In certain embodiments, when X¹¹ and X³³ are —CH₂—, X²² is —CR^{2a}—; L¹, L², and L³ are —OC(O)—; R^{2a} and R² together with atoms attached thereto are joined to form a

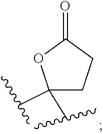 [SMILES: *C1(*)CCC(=O)O1]

and R¹ is C₁-C₆, C₈, C₁₁, C₁₃, C₁₅, C₁₉ unsubstituted alkyl, cyclopropyl, or cyclohexyl, then R³ is not

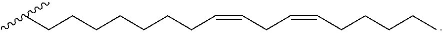 [SMILES: *CCCCCCC/C=C\C/C=C\CCCCC]

In certain embodiments, when X¹¹ and X³³ are —CH₂—, X²² is —CR^{2a}—; L¹, L², and L³ are —OC(O)—; R^{2a} and R² together with atoms attached thereto are joined to form a

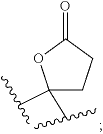 [SMILES: *C1(*)CCC(=O)O1]

and R¹ is methyl, C₃-C₄, C₁, C₁₃ unsubstituted alkyl, cyclopropyl, or cyclohexyl, then R³ is not unsubstituted C₁₅ alkyl.

In certain embodiments, when X¹¹, X²², X³³ are —CH₂—; L¹, L², and L³ are —OC(O)—; R¹ is C₁₅ unsubstituted alkyl; and R³ is unsubstituted C₁-C₄ alkyl, C₆, C₇, phenyl, cyclopropyl, cyclohexyl, or —CH₂—NH₂, then R² is not

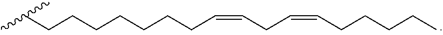 [SMILES: *CCCCCCC/C=C\C/C=C\CCCCC]

In certain embodiments, when X¹¹, X²², X³³ are —CH₂—; L¹ and L² are —OC(O)—; L³ is —O—; R³ is unsubstituted C₁-C₃ alkyl, or —CH(CH₃)—OCH₃; R¹ is C₇, C₉, C₁₁ unsubstituted alkyl; and R³ is C₂-C₄, then R² is not

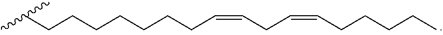 [SMILES: *CCCCCCC/C=C\C/C=C\CCCCC]

In certain embodiments, when X¹¹, X²², X³³ are —CH₂—; L¹ and L² is —OC(O)—; and L³ is —NHC(O)— or —SC(O)—; and R³ is methyl, then R² is not

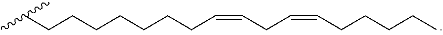 [SMILES: *CCCCCCC/C=C\C/C=C\CCCCC]

In certain embodiments, when X¹¹, X²², X³³ are —CH₂—; L¹ and L² is —OC(O)—; and L³ is —O—; and R³ is methyl or ethyl, then R² is not

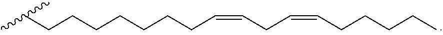 [SMILES: *CCCCCCC/C=C\C/C=C\CCCCC]

In certain embodiments, when X¹¹, X²², X³³ are —CH₂—; L¹ is —NHC(O)—; L² and L³ is —OC(O)—, R¹ is C₁₅ unsubstituted alkyl; R² is C₁-C₄ alkyl, cyclopropyl, or cyclohexyl, then R³ is not

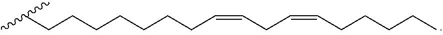 [SMILES: *CCCCCCC/C=C\C/C=C\CCCCC]

In certain embodiments, when X¹¹, X²², X³³ are —CH₂—; L¹ is —NHC(O)—; L² and L³ is —OC(O)—, R¹ is C₁₅ unsubstituted alkyl; R³ is methyl, ethyl, propyl, 2-methyl propyl, or cyclopropyl, then R² is not

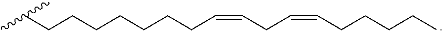 [SMILES: *CCCCCCC/C=C\C/C=C\CCCCC]

In certain embodiments, when X¹¹, X²², X³³ are —CH₂—; each L¹, L² and L³ are —NHC(O)—; —O—, —SC(O), —OC(O)—, R¹ is C₁₅ unsubstituted alkyl; R³ is hydrogen or methyl, then R² is not

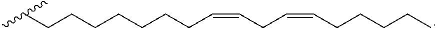 [SMILES: *CCCCCCC/C=C\C/C=C\CCCCC]

In certain embodiments, when X¹¹, X²², X³³ are —CH₂—; each L¹, L² and L³ are —NHC(O)—; —O—, —SC(O), —OC(O)—, one of R² and R³ is C₁₅ unsubstituted alkyl; and the other of R² and R³ is hydrogen, then R¹ is not

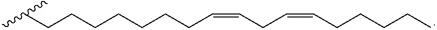 [SMILES: *CCCCCCC/C=C\C/C=C\CCCCC]

In certain embodiments, when X¹¹, X²², X³³ are —CH₂—; L¹ is —OC(O)—, R¹ is methyl or unsubstituted C₁₅ alkyl, L² is —O—, and R² is hydrogen, then L³-R³ is not OH or —OC(O)CH₃.

In certain embodiments, when X¹¹ and X²² are —CH₂—; X³³ is —C(O)—; L¹ and L₂ are —OC(O)—; one of R¹ and R² is unsubstituted C₁₅ alkyl; the other of R¹ and R² is

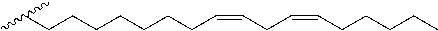 [SMILES: *CCCCCCC/C=C\C/C=C\CCCCC]

and L³ is —NH—; then R³ is not —CH₂—CH₃.

In certain embodiments, when X¹¹ and X²² are —CH₂—; X³³ is —C(O)—; L¹ and L₂ are —OC(O)—; R² is unsubstituted C₁₅ alkyl; R¹ is

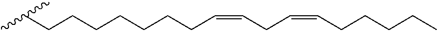 [SMILES: *CCCCCCC/C=C\C/C=C\CCCCC]

and L³ is —O—; then R³ is not —CH₂—CH₃.

In certain embodiments, when X¹¹, X²², X³³ are —CH₂—; L¹, L² and L³ are —OC(O)—; R¹ and R² are same as C₁₅ alkyl or

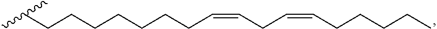 [SMILES: *CCCCCCC/C=C\C/C=C\CCCCC]

then R³ is not —CH₃.

In certain embodiments, when X¹¹, X²², X³³ are —CH₂—; L¹, L² and L³ are —OC(O)—R¹, R², R³ are not the same as unsubstituted C₇ or C₉ alkyl.

In some embodiments, R^{2a} and R² together with atoms attached thereto are joined to form a substituted or unsubstituted C₅-C₈ cycloalkyl, substituted or unsubstituted 5 to 8 membered heterocycloalkyl.

In one preferred embodiments, the compound has the structure of: the compound has the structure of:

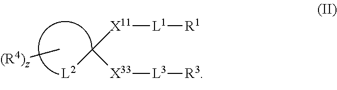

In formula (II), L² is a bond, —C(O)—, —OC(O)—, —SC(O)—, —C(O)O—, —C(O)S—, —NHC(O)—, —C(O)NH—, —NHC(O)NH—, —NH—, —NCH₃—, —O—, —S—, —S(O)₂—, —NHS(O)₂—, —S(O)₂NH—, —NHC(O)O—, —OC(O)NH, substituted or unsubstituted C₁-C₃ alkylene, or substituted or unsubstituted 2 to 3 membered heteroalkylene;

z is an integer from 0 to 8;

R⁴ is halogen, —CX⁴₃, —CHX⁴₂, —CH₂X⁴, —OCX⁴₃, —OCH₂X⁴, —OCHX⁴₂, —N₃, —CN, —SO₂R^{4D}, —SO₂NR^{4A}R^{4B}, —NHC(O)NR^{4A}R^{4B}, —NO₂, —NR^{4A}R^{4B}, —C(O)R^{4C}, —C(O)—OR^{4C}, —C(O)NR^{4A}R^{4B}, —OR^{4D}, —NR^{4A}SO₂R^{4D}, —NR^{4A}C(O)R^{4C}, —NR^{4A}C(O)OR^{4C}, —NR^{4A}OR^{4C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

R^{4A}, R^{4B}, R^{4C}, and R^{4D} are independently hydrogen, —CX₃, —CHX₂, —CH₂X, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;

X⁴ is independently —F, Cl, —Br, or —I.

In some embodiment, z is 0. In some embodiments, z is 1. In some embodiments, z is 2. In some embodiments, z is 3. In some embodiments, z is 4.

In some embodiments, the compound has the structure of:

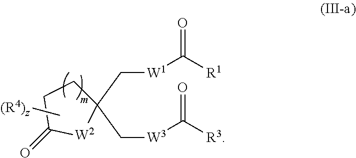

In formula (III-a), each W¹, W², and W³ is independently —NH—, —O—, or —S—;

m is an integer from 0 to 4.

In some embodiments, m is 0. In some embodiments, m is 1. In some embodiments, m is 2.

In certain embodiments, the compound has the structure of:

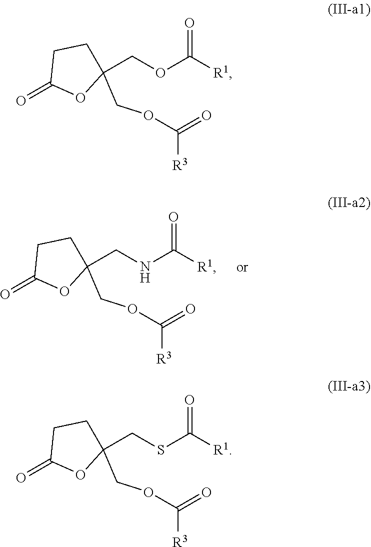

In certain embodiments, the compound has the structure of:

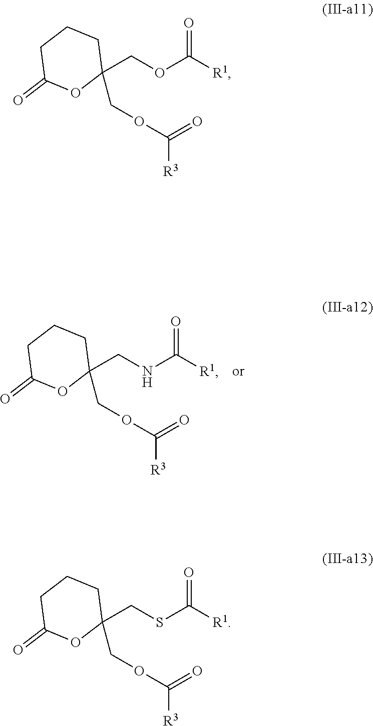

In some embodiments, the compound has the structure of:

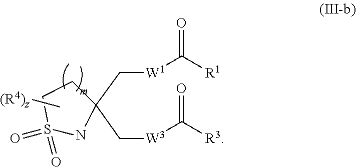

In formula (III-b), W¹, W², and W³ is independently —NH—, —O—, or —S—;

m is an integer from 0 to 4.

In certain embodiments, the compound has the structure of:

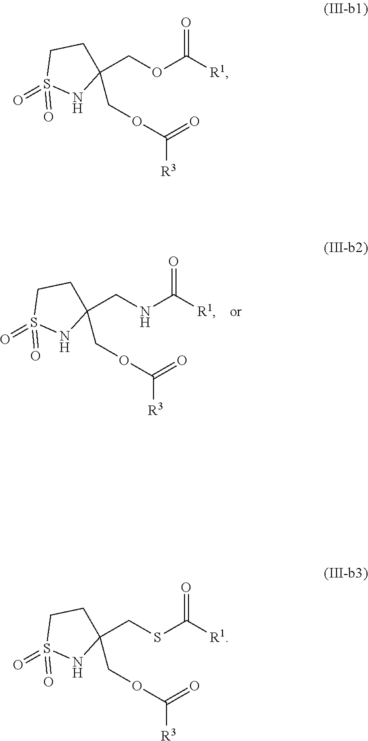

In certain embodiments, the compound has the structure of:

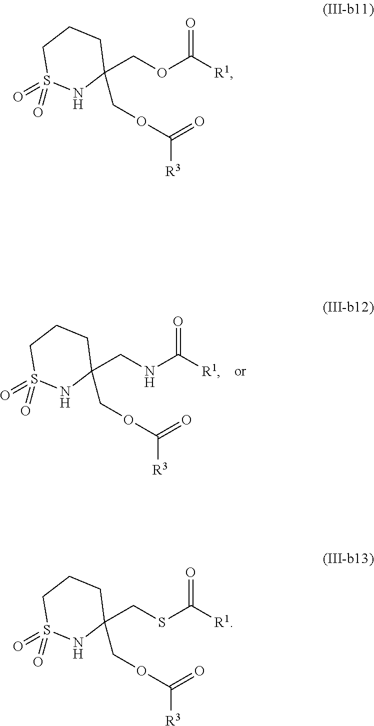

In some embodiments, X³³ is —C(O)—. In some embodiments, the compound has the structure of:

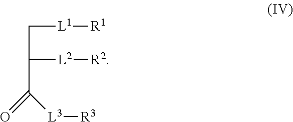

R¹, R² and R³ are as described above.

In formula (IV), each L¹, L², and L³ is independently a bond, —OC(O)—, —SC(O)—, or —NHC(O)—.

In some embodiments, L² is a bond. In some embodiments, L³ is a bond.

In some embodiments, the compound has the structure of:

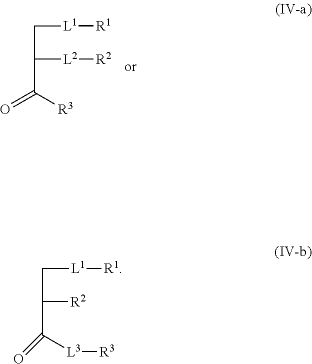

L¹, L², R¹, R² and R³ are as described above.

In some embodiments, L³ is —NR¹³C(O)—. In some embodiments, R² and R¹³ are joined to form a substituted or unsubstituted 5-8 membered heterocycloalkyl. In some embodiments, R² and R¹³ are joined to form

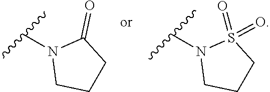 [SMILES: *N1CCCC1=O ; *N1CCCS1(=O)=O]

In some embodiments, L³ is —NR¹⁵C(O)—. In some embodiments, R³ and R¹⁵ are joined to form a substituted or unsubstituted 5-8 membered heterocycloalkyl. In some embodiments, R³ and R¹⁵ are joined to form

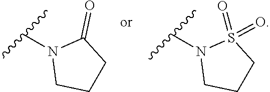 [SMILES: *N1CCCC1=O ; *N1CCCS1(=O)=O]

In some embodiments, the compound has the structure of:

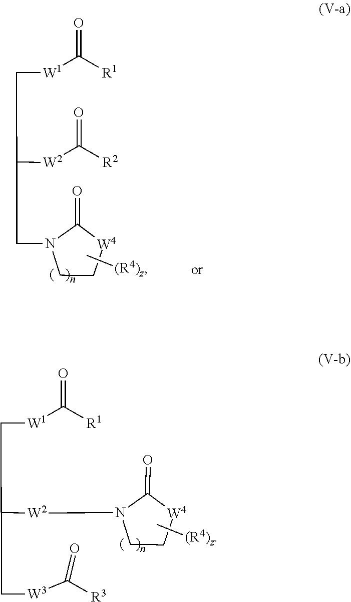

R¹, R² and R³ are as described above.

In formula (V-a) or (V-b), each W¹ and W² is independently a bond, —NH—, —O—, or —S—. W⁴ is —NR¹—, —CH₂—, —O—. n is an integer from 0 to 4. z is an integer from 0 to 8. R⁴ is halogen, —CX⁴₃, —CHX⁴₂, —CH₂X⁴, —OCX⁴₃, —OCH₂X⁴, —OCHX⁴₂, —N₃, —CN, —SO₂R^{4D}, —SO₂NR^{4A}R^{4B}, —NHC(O)NR^{4A}R^{4B}, —NO₂, —NR^{4A}R^{4B}, —C(O)R^{4C}, —C(O)—OR^{4C}, —C(O)NR^{4A}R^{4B}, —OR^{4D}, —NR^{4A}SO₂R^{4D}, —NR^{4A}C(O)R^{4C}, —NR^{4A}C(O)OR^{4C}, —NR^{4A}OR^{4C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R^{4A}, R^{4B}, R^{4C}, and R^{4D} are independently hydrogen, —CX₃, —CHX₂, —CH₂X, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. X⁴ is independently —F, Cl, —Br, or —I.

For example, the compound has the structure of:

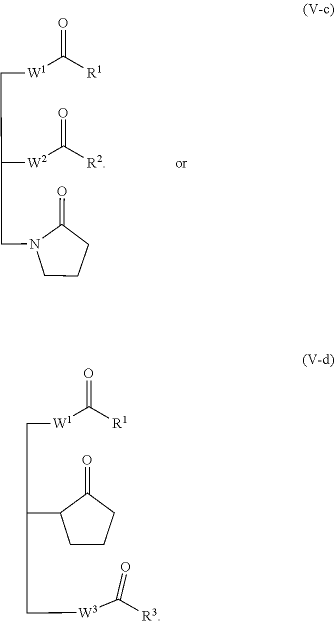

described above.

For example, the compound has the structure of:

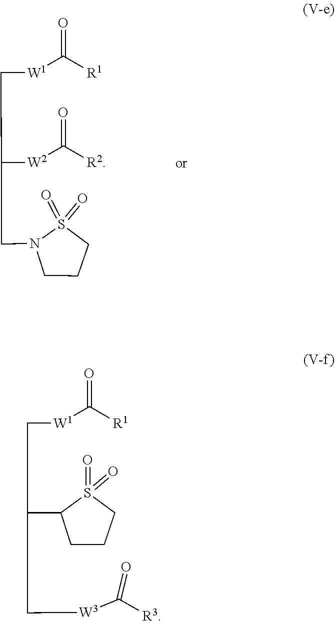

R¹, R² and R³ are as described above.

In preferred embodiments, R¹ is hydrogen, R^{1E}-substituted or unsubstituted C₁-C₂₀ alkyl, and R^{1E} is halogen, —OH, —NH₂, —COOH, —NO₂, —N₃, —CN, substituted or unsubstituted phenyl, a cholesterol or its derivative, a carbohydrate, —P(O)₂OH, —P(O)(OH)₂, a nucleic acid, or a peptide.

In some embodiments, R¹ is unsubstituted saturated C₁-C₂₀ alkyl.

In some embodiments, R¹ is unsubstituted saturated unbranched C₁-C₂₀ alkyl.

In some embodiments. R¹ is unsubstituted unsaturated C₁-C₂₀ alkyl.

In some embodiments, R¹ is unsubstituted unsaturated unbranched C₁-C₂₀ alkyl.

In some embodiments, R¹ is unsubstituted unsaturated C₁₀-C₂₀ alkyl.

In some embodiments, R¹ is unsubstituted unsaturated unbranched C₁₀-C₂₀ alkyl.

In some embodiments, R¹ is unsubstituted unsaturated unbranched C₁₀-C₂₀ alkyl.

In some embodiments, R¹ is unsubstituted saturated C₁-C₁₀ alkyl.

In some embodiments, R¹ is unsubstituted saturated C₁-C₄ alkyl.

For example, R¹ is methyl, ethyl, propyl, isopropyl, 2-methyl propyl, butyl, isobutyl, or t-butyl.

In some embodiments, R¹ is unsubstituted C₃-C₆ cycloalkyl. For example, R¹ is unsubstituted cyclopropyl or cyclohexyl. In some embodiments, R¹ is unsubstituted aryl (e.g., phenyl).

In preferred embodiments, R² is hydrogen, R^{2E}-substituted or unsubstituted C₁-C₂₀ alkyl, and R^{2E} is halogen, —OH, —NH₂, —COOH, —NO₂, —N₃, —CN, substituted or unsubstituted phenyl, a cholesterol or its derivative, a carbohydrate, —P(O)₂OH, —P(O)(OH)₂, a nucleic acid, or a peptide.

In some embodiments, R² is unsubstituted saturated C₁-C₂₀ alkyl.

In some embodiments, R² is unsubstituted saturated unbranched C₁-C₂₀ alkyl.

In some embodiments, R² is unsubstituted unsaturated C₁-C₂₀ alkyl.

In some embodiments, R² is unsubstituted unsaturated unbranched C₁-C₂₀ alkyl.

In some embodiments, R² is unsubstituted unsaturated C₁₀-C₂₀ alkyl.

In some embodiments, R² is unsubstituted unsaturated unbranched C₁₀-C₂₀ alkyl.

In some embodiments, R² is unsubstituted unsaturated unbranched C₁₀-C₂₀ alkyl.

In some embodiments, R² is unsubstituted saturated C₁-C₁₀ alkyl.

In some embodiments, R² is unsubstituted saturated C₁-C₄ alkyl.

For example, R² is methyl, ethyl, propyl, isopropyl, 2-methyl propyl, butyl, isobutyl, or t-butyl.

In some embodiments, R² is unsubstituted C₃-C₆ cycloalkyl. For example, R² is unsubstituted cyclopropyl or cyclohexyl. In some embodiments, R² is unsubstituted aryl (e.g., phenyl).

In preferred embodiments, R³ is hydrogen, R^{3E}-substituted or unsubstituted C₁-C₂₀ alkyl, R^{3E}-substituted or unsubstituted 2 to 10 membered heteroalkyl, R^{3E}-substituted or unsubstituted C₃-C₈ cycloalkyl, or R^{3E}-substituted or unsubstituted phenyl, and R^{3E} is halogen, —OH, —NH₂, —COOH, —NO₂, —N₃, —CN, substituted or unsubstituted phenyl, a cholesterol or its derivative, a carbohydrate, —P(O)₂OH, —P(O)(OH)₂, a nucleic acid, or a peptide.

In some embodiments, R³ is unsubstituted saturated C₁-C₂₀ alkyl.

In some embodiments, R³ is unsubstituted saturated unbranched C₁-C₂₀ alkyl.

In some embodiments, R³ is unsubstituted unsaturated C₁-C₂₀ alkyl.

In some embodiments, R³ is unsubstituted unsaturated unbranched C₁-C₂₀ alkyl.

In some embodiments, R³ is unsubstituted unsaturated C₁₀-C₂₀ alkyl.

In some embodiments, R³ is unsubstituted unsaturated unbranched C₁₀-C₂₀ alkyl.

In some embodiments, R³ is unsubstituted unsaturated unbranched C₁₀-C₂₀ alkyl.

In some embodiments, R³ is unsubstituted saturated C₁-C₁₀ alkyl.

In some embodiments, R³ is unsubstituted saturated C₁-C₁₀ alkyl.

In some embodiments, R³ is unsubstituted saturated C₁-C₄ alkyl.

For example, R³ is methyl, ethyl, propyl, isopropyl, 2-methyl propyl, butyl, isobutyl, or t-butyl.

In some embodiments, R³ is unsubstituted C₃-C₆ cycloalkyl. For example, R³ is unsubstituted cyclopropyl or cyclohexyl. In some embodiments, R³ is unsubstituted aryl (e.g., phenyl).

In some embodiments, one of R¹, R², and R³ is unsubstituted C₃-C₆ cycloalkyl (e.g. cyclopropyl or cyclohexyl).

In some embodiment, the compound is

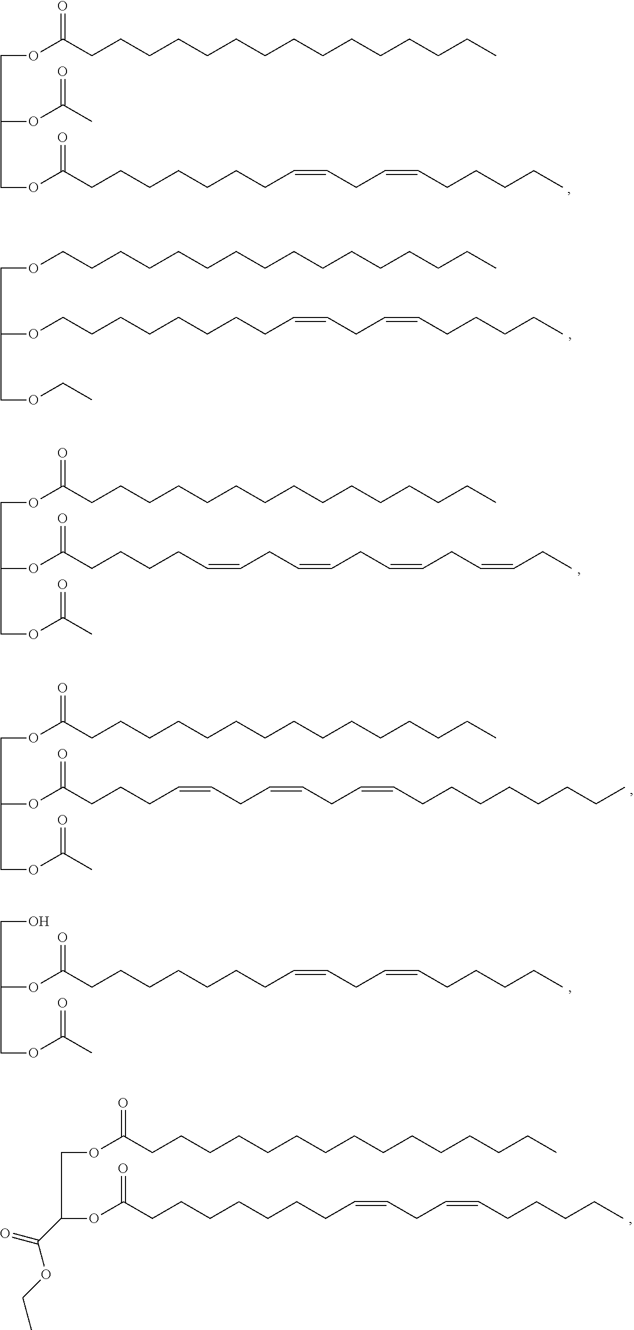 [SMILES: CCCCC/C=C\C/C=C\CCCCCCCC(=O)OCC(COC(=O)CCCCCCCCCCCCCCC)OC(C)=O ; CCCCC/C=C\C/C=C\CCCCCCCCOC(COCC)COCCCCCCCCCCCCCCCC ; CC/C=C\C/C=C\C/C=C\C/C=C\CCCCC(=O)OC(COC(C)=O)COC(=O)CCCCCCCCCCCCCCC ; CCCCCCCC/C=C\C/C=C\C/C=C\CCCC(=O)OC(COC(C)=O)COC(=O)CCCCCCCCCCCCCCC ; CCCCC/C=C\C/C=C\CCCCCCCC(=O)OC(CO)COC(C)=O ; CCCCC/C=C\C/C=C\CCCCCCCC(=O)OC(COC(=O)CCCCCCCCCCCCCCC)C(=O)OCC]

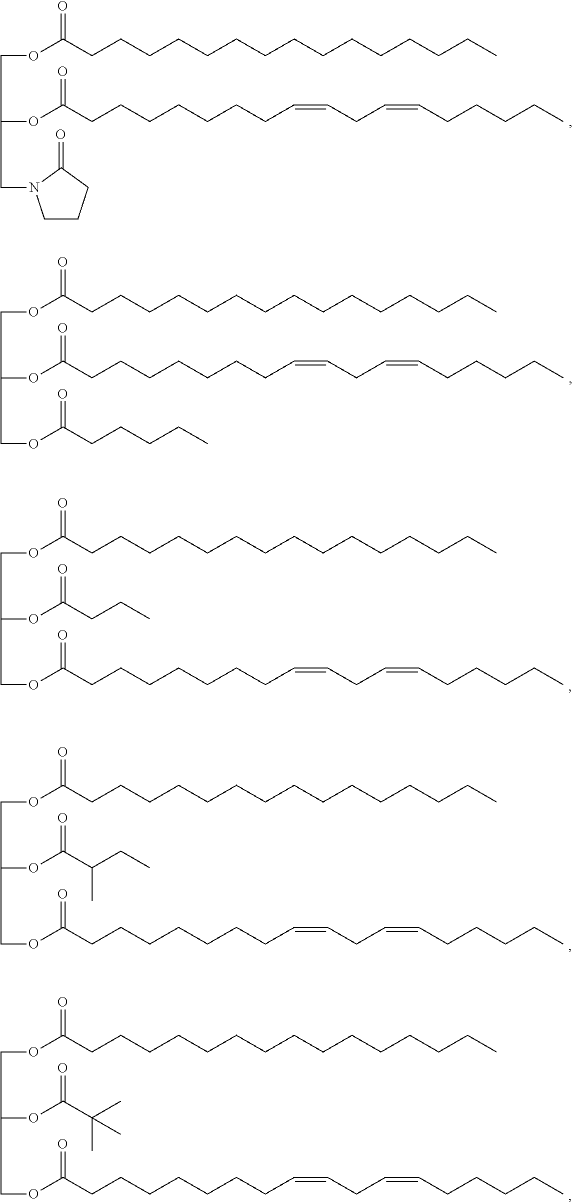

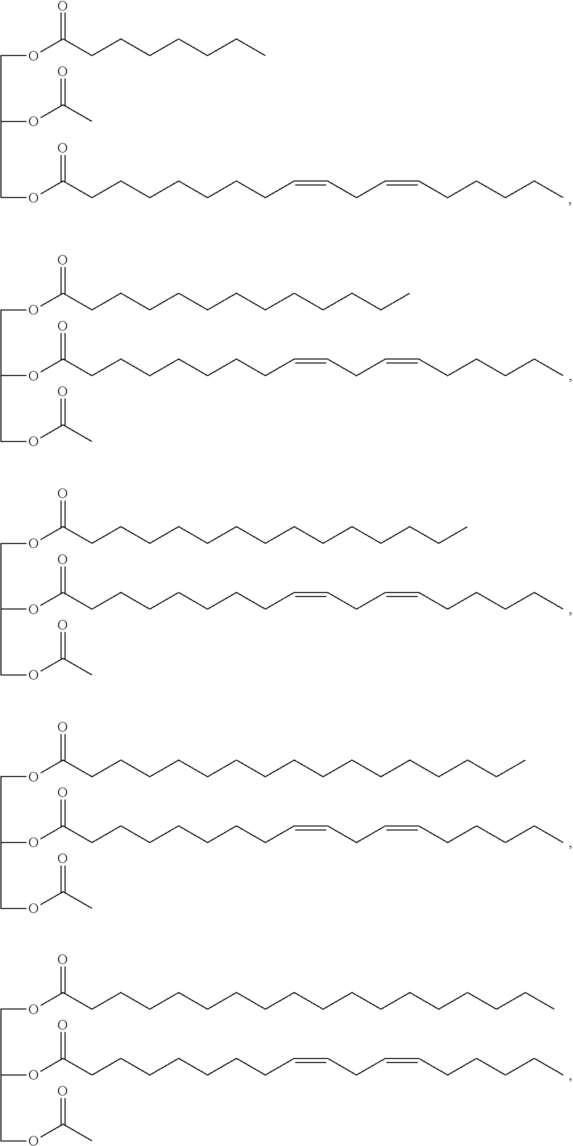 [SMILES: CCCCC/C=C\C/C=C\CCCCCCCC(=O)OCC(COC(=O)CCCCCCC)OC(C)=O ; CCCCC/C=C\C/C=C\CCCCCCCC(=O)OC(COC(C)=O)COC(=O)CCCCCCCCCCCC ; CCCCC/C=C\C/C=C\CCCCCCCC(=O)OC(COC(C)=O)COC(=O)CCCCCCCCCCCCCC ; CCCCC/C=C\C/C=C\CCCCCCCC(=O)OC(COC(C)=O)COC(=O)CCCCCCCCCCCCCCCC ; CCCCC/C=C\C/C=C\CCCCCCCC(=O)OC(COC(C)=O)COC(=O)CCCCCCCCCCCCCCCCC]

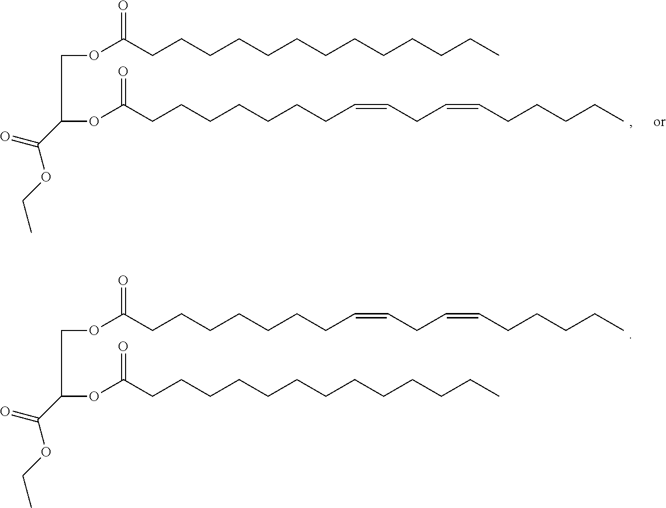 [SMILES: CCCCC/C=C\C/C=C\CCCCCCCC(=O)OC(COC(=O)CCCCCCCCCCCCC)C(=O)OCC ; CCCCC/C=C\C/C=C\CCCCCCCC(=O)OCC(OC(=O)CCCCCCCCCCCCC)C(=O)OCC]

### III. Compositions

As discussed, one aspect of the present invention provides a therapeutic pharmaceutical composition including the compound as describe herein.

The composition may be administered as an individual therapeutic agent or may be administered in combination with another drug that is known to have an efficacy on treating the particular indication. For example, the above composition may be administered with one or more of therapeutic agents including proteins, small molecule drugs, nucleic acids or the like. For example, the composition may be administered with a therapeutic agent including granulocyte-colony stimulating factor (G-CSF), but the administration is not limited thereto. Further, the above composition can be administered together with analgesics, anti-ulcer agents, antidiarrheic, antibiotics, antipyretics, nutritional supplements and antioxidants, which can help preventing or treating a desired indication.

The term “administration” in the present invention means introducing a therapeutic pharmaceutical composition of the present invention to a patient by any suitable method, and the administration route of the composition of the present invention may be administered via various routes whether orally or non-orally. The therapeutic pharmaceutical composition of the present invention can be manufactured into various formulations depending on the administration methods.

The frequency of administration of the composition of the present invention is not particularly limited, but it may be administered once a day or several times a day with divided dosage.

The therapeutic pharmaceutical composition of the present invention can be used as a single medication, and can be used as a combined medication containing another drug, and can be formulated with using a pharmaceutically acceptable carrier, excipient or diluent to make a single-dose unit or a unit with a multi-dose container.

The term “pharmaceutical composition” as referred to herein indicates a composition prepared for the purpose of preventing or treating diseases, and can be formulated into various forms according to ordinary methods. For example, it can be formulated into oral administration formulations such as powders, granules, tablets, capsules, suspensions, emulsions and syrups, and can be formulated in the form of external use, suppositories, and sterilized injection solutions.

In addition, the pharmaceutical composition of the present invention may be manufactured with additional pharmaceutically acceptable carrier for each formulation. As used herein, the term “pharmaceutically acceptable carrier” may refer to a carrier or diluent that does not stimulate organism and not inhibiting biological activity and characteristic of the injected compound. The type of the carrier that can be used in the present invention is not particularly limited, any carrier conventionally used in the area of industry and pharmaceutically acceptable may be used.

Saline, sterilized water, IV fluids, buffer saline, albumin injection solution, dextrose solution, maltodextrin solution, glycerol, ethanol are non-limiting examples of the usable carriers. These carriers may be used alone or in combination of two or more. The carrier may include a non-naturally occurring carrier. If necessary, other conventionally used additives like an antioxidant, a buffer and/or a bacteriostatic agent may be added and used. It may be formulated with diluent, a dispersant, a surfactant, a bonding agent, a lubricant to make an injection solution like aqueous solution, suspension, emulsion, and pills, capsules, granules or tablets, and the like.

In addition, the pharmaceutical composition of the present invention may contain a pharmaceutically effective amount of the compounds as described herein. The term “pharmaceutically effective amount” in the present invention means an amount sufficient to treat a disease at a reasonable benefit or risk ratio applicable to medical treatment and is generally in the range of about 0.001 to 5000 mg/kg, preferably of about 0.05 to 1000 mg/kg, may be administered once a day or several times a day with divided dosage. However, for the purposes of the present invention, the specific therapeutically effective amount for a particular patient will depend upon the nature and extent of the reaction to be achieved, the particular composition, including whether or not other agents are used, the age, weight, sex and diet of the patient, the time of administration, the route of administration and the proportion of the composition, the duration of the treatment, the drugs administered or co-administered with the specific composition, and similar compounds well known in the medical industry.

As discussed, kits are also provided. For instance, in this aspect, a compound as described herein suitably can be packaged in suitable containers labeled, for example, for use as a therapy to treat a subject suffering from cancer, or acute radiation syndrome, or inflammation, or a subsyndrome thereof. The containers can include a compound as described herein or composition and one or more of a suitable stabilizer, carrier molecule and/or the like, as appropriate for the intended use. In other embodiments, the kit further comprises one or more therapeutic reagents that alleviate some of the symptoms or secondary infections or disorders that may be associated with cancer, acute radiation syndrome or inflammation.

Accordingly, packaged products (e.g., sterile containers containing one or more of the compositions described herein and packaged for storage, shipment, or sale at concentrated or ready-to-use concentrations) and kits, including a compound as described herein, and instructions for use, are also within the scope of the invention. A product can include a container (e.g., a vial, jar, bottle, bag, or the like) containing a compound as described herein or composition. In addition, an article of manufacture or kit further may include, for example, packaging materials, instructions for use, syringes, delivery devices, for treating or monitoring the condition for which prophylaxis or treatment is required.

The product may also include a legend (e.g., a printed label or insert or other medium describing the product's use (e.g., an audio- or videotape)). The legend can be associated with the container (e.g., affixed to the container) and can describe the manner in which the compositions therein should be administered (e.g., the frequency and route of administration), indications therefor, and other uses. The compositions can be ready for administration (e.g., present in dose-appropriate units), and may include one or more additional pharmaceutically acceptable adjuvants, carriers or other diluents and/or an additional therapeutic agent. Alternatively, the compositions for example can be provided in a concentrated form with a diluent and instructions for dilution.

Another aspect of the present invention is a health functional food composition of food supplement comprising compounds as described herein as an active ingredient.

In the present invention, the term “improvement” means all actions that at least reduce the degree of symptom associated with the condition being treated. Herein, the health functional food composition may be used simultaneously or separately with the medicament for treatment before or after the occurrence of the disease to prevent or improve the targeted disease or disorder.

Functional food is the same term as food for special health use (FoSHU). It refers to foods that have been processed so that the biological control function appears more efficient in addition to nutritional value. The food may be prepared in various forms such as tablets, capsules, powders, granules, liquids, rings and the like in order to obtain a useful effect on skin regeneration.

For that, the content level of the compound as described herein contained in the health functional food is not particularly limited, but may be 0.01 to 100% by weight, specifically 1 to 80% by weight based on the total weight of the health functional food.

The health functional food composition of the present invention may also contain a pharmaceutically acceptable carrier.

There is no particular limitation on the kind of health functional foods including the compound as described herein, and examples thereof include drinks, gums, tea, vitamin complex, health supplement foods and the like. The food may be supplemented with other ingredients that do not interfere with the improvement effect on the targeted disease or disorder, and the kind thereof is not particularly limited. For example, various herbal extracts, sitology-acceptable food supplementary or other natural carbohydrates may be added as an additional ingredient.

The food-aid additive described above is added to produce the health functional food of each formulation and can be appropriately selected and used by a person skilled in the relevant field of technology. For example, various nutrient additives, vitamins, minerals (electrolytes), synthetic flavors and natural flavors, colorants and fillers, pectic acid and its salts, alginic acid and its salts, organic acids, protective colloid thickeners, pH controller, stabilizer, preservative, glycerin, alcohol, carbonating agent used in a carbonated drink, and the like, but the kind is not limited by the above.

In addition, the health functional food described above may contain additional ingredients which are commonly used in food to improve smell, taste, visual appearance and the like. For example, vitamins A, C, D, E, B1, B2, B6, B12, niacin, biotin, folate, panthotenic acid and the like can be included. In addition, it may include minerals such as zinc (Zn), iron (Fe), calcium (Ca), chromium (Cr), magnesium (Mg), manganese (Mn) and copper (Cu) and the like. It may also contain amino acids such as lysine, tryptophan, cysteine, valine and the like.

In addition, the described health functional food may include one or more preservatives (such as potassium sorbate, sodium benzoate, salicylic acid, and sodium dehydroacetate), bactericides (such as bleaching powder and high bleaching powder, sodium hypochlorite), antioxidants (butylhydroxyanilide (BHA), butylhydroxytoluene (BHT), etc.), coloring agents (such as tar pigments), color formers (such as sodium nitrite and sodium acetates), bleaching agents (sodium sulfite), seasonings (such MSG, sodium glutamate), sweeteners (such as dulcin, cyclamate, saccharin, sodium), flavorings (vanillin, lactones, etc.), swelling agents (alum, potassium hydrogen D-tartrate), fortifier, emulsifiers, thickeners, encapsulating agents, gum bases, foam inhibitors, solvent, improver, and the like. The above additives are selected according to the type of food and used in an appropriate amount.

The health functional food composition of the present invention can be prepared by a method commonly used in the industry and can be prepared by adding raw materials and ingredients which are conventionally added in the industry. In addition, unlike general medicine, the health functional food may have an advantage, fo example, as there can be no side effect from a long-term use and have better portability.

The pharmaceutical composition may be prepared and administered in a wide variety of dosage formulations. Compounds described may be administered orally, rectally, or by injection (e.g. intravenously, intramuscularly, intracutaneously, subcutaneously, intraduodenally, or intraperitoneally).

For preparing pharmaceutical compositions from compounds described herein, pharmaceutically acceptable carriers can be either solid or liquid. Solid form preparations include powders, tablets, pills, capsules, cachets, suppositories, and dispersible granules. A solid carrier may be one or more substance that may also act as diluents, flavoring agents, binders, preservatives, tablet disintegrating agents, or an encapsulating material.

In powders, the carrier may be a finely divided solid in a mixture with the finely divided active component. In tablets, the active component may be mixed with the carrier having the necessary binding properties in suitable proportions and compacted in the shape and size desired.

The powders and tablets preferably contain from 5% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations that are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.1 mg to 10000 mg according to the particular application and the potency of the active component. The composition can, if desired, also contain other compatible therapeutic agents.

Some compounds may have limited solubility in water and therefore may require a surfactant or other appropriate co-solvent in the composition. Such co-solvents include: Polysorbate 20, 60, and 80; Pluronic F-68, F-84, and P-103; cyclodextrin; and polyoxyl 35 castor oil. Such co-solvents are typically employed at a level between about 0.01% and about 2% by weight. Viscosity greater than that of simple aqueous solutions may be desirable to decrease variability in dispensing the formulations, to decrease physical separation of components of a suspension or emulsion of formulation, and/or otherwise to improve the formulation. Such viscosity building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose, chondroitin sulfate and salts thereof, hyaluronic acid and salts thereof, and combinations of the foregoing. Such agents are typically employed at a level between about 0.01% and about 2% by weight.

The pharmaceutical compositions may additionally include components to provide sustained release and/or comfort. Such components include high molecular weight, anionic mucomimetic polymers, gelling polysaccharides, and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes.

The pharmaceutical composition may be intended for intravenous use. The pharmaceutically acceptable excipient can include buffers to adjust the pH to a desirable range for intravenous use. Many buffers including salts of inorganic acids such as phosphate, borate, and sulfate are known.

### Effective Dosages

The pharmaceutical composition may include compositions wherein the active ingredient is contained in a therapeutically effective amount, i.e., in an amount effective to achieve its intended purpose. The actual amount effective for a particular application will depend, inter alia, on the condition being treated.

The dosage and frequency (single or multiple doses) of compounds administered can vary depending upon a variety of factors, including route of administration; size, age, sex, health, body weight, body mass index, and diet of the recipient; nature and extent of symptoms of the disease being treated; presence of other diseases or other health-related problems; kind of concurrent treatment; and complications from any disease or treatment regimen. Other therapeutic regimens or agents can be used in conjunction with the methods and compounds disclosed herein.

Dosages may be varied depending upon the requirements of the subject and the compound being employed. The dose administered to a subject, in the context of the pharmaceutical compositions presented herein, should be sufficient to effect a beneficial therapeutic response in the subject over time. The size of the dose also will be determined by the existence, nature, and extent of any adverse side effects. Generally, treatment is initiated with smaller dosages, which are less than the optimum dose of the compound. Thereafter, the dosage is increased by small increments until the optimum effect under circumstances is reached.

Dosage amounts and intervals can be adjusted individually to provide levels of the administered compounds effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is entirely effective to treat the clinical symptoms demonstrated by the particular patient. This planning should involve the careful choice of active compound by considering factors such as compound potency, relative bioavailability, patient body weight, presence and severity of adverse side effects, preferred mode of administration, and the toxicity profile of the selected agent.

### Toxicity

The ratio between toxicity and therapeutic effect for a particular compound is its therapeutic index and can be expressed as the ratio between LD₅₀ (the amount of compound lethal in 50% of the population) and ED₅₀ (the amount of compound effective in 50% of the population). Compounds that exhibit high therapeutic indices are preferred. Therapeutic index data obtained from cell culture assays and/or animal studies can be used in formulating a range of dosages for use in humans. The dosage of such compounds preferably lies within a range of plasma concentrations that include the ED₅₀ with little or no toxicity. The dosage may vary within this range depending upon the dosage form employed and the route of administration utilized. See, e.g. Fingl et al., In: THE PHARMACOLOGICAL BASIS OF THERAPEUTICS, Ch. 1, p. 1, 1975. The exact formulation, route of administration, and dosage can be chosen by the individual physician in view of the patient's condition and the particular method in which the compound is used.

When parenteral application is needed or desired, particularly suitable admixtures for the compounds included in the pharmaceutical composition may be injectable, sterile solutions, oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-block polymers, and the like. Ampoules are convenient unit dosages. Pharmaceutical admixtures suitable for use in the pharmaceutical compositions presented herein may include those described, for example, in Pharmaceutical Sciences (17th Ed., Mack Pub. Co., Easton, PA) and WO 96/05309, the teachings of both of which are hereby incorporated by reference.

### IV. Methods of Treatment

As discussed, further provided is a method of treating a patient suffering from cancer, including administering a compound as described herein to a patient that is suffering from cancer, including a solid tumor.

Methods are also provided to treat a patient suffering from or susceptible to acute radiation syndrome, including administering a compound as described herein to a patient that is suffering from or susceptible to acute radiation syndrome.

Methods are further provided to treat a subject that has been exposed to ionizing radiation (particularly adverse exposure such as unintended and/or non-therapeutic exposure, and/or exposure to excessive ionizing radiation, including gamma radiation) which include administering to the subject an effective amount of a compound disclosed herein.

In a yet further aspect, methods are also provided to treat a patient suffering from or susceptible to mucosits, including oral mucositis (e.g., oral ulceration) or gastrointestinal mucositis, including administering a compound as described herein to a patient that is suffering from or susceptible to mucositis, including oral mucositis (e.g. oral ulceration) or gastrointestiial mucositis.

### V. Examples

Although the foregoing section has been described in some detail by way of illustration and example for purposes of clarity of understanding, it is apparent to those skilled in the art that certain minor changes and modifications will be practiced in light of the above teaching. Therefore, the description and examples should not be construed as limiting the scope of any invention described herein.

### Example 1

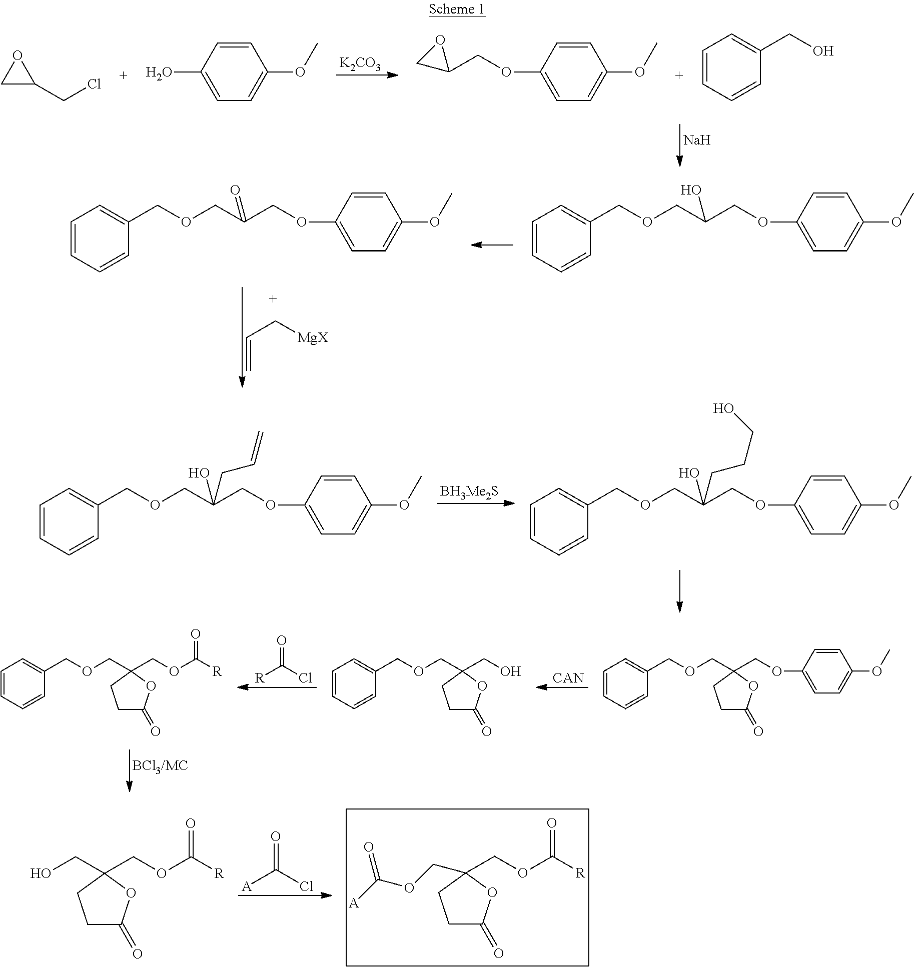

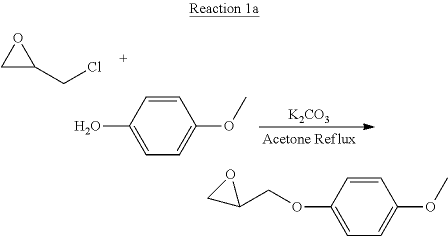

As shown in the above Reaction 1a, after dissolving 50 g (402.77 mmole) of 4-methyoxyphenol in 1500 ml of acetone, 278 g (2013.8 mmole) of K₂CO₃ was added to the mixture and stirred at room temperature for 30 minutes. 126 ml (1611.1 mmole) of epichlorohydrin was added to that 4-methyoxyphenol mixture solution and refluxed at a temperature of 60 to 65° C. for 72 hours. The reaction was monitored by TLC (EA:Hex=1:9). When the reaction was completed, the reaction mixture was filtered by Celite filter and concentrated. The concentrate was purified by flash column chromatography using mixed solution of (EA:Hex)=1:10 (volume ratio) to afford 69 g of the target compound at yield of 93.7%.

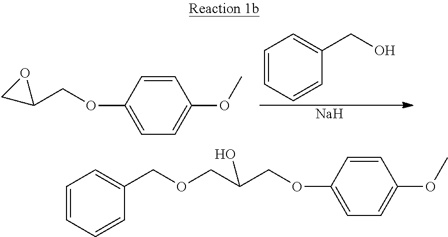

As shown in the above Reaction 1b, after dissolving benzylacohol 0.73 ml (7.0755 mmole) in 4 ml of dimethylformamide, DMF, 60%-NaH 283 mg (7.0755 mmole) was slowly added and stirred at inner room temperature for 30 minutes. A solution prepared by dissolving 850 mg (4.717 mmole) of the product (SM) of the Reaction 1a in 3 ml of dime was slowly dropped in the reaction solution and stirred at a temperature of 80° C. for 3 hours. The reaction was monitored as TLC (EA:Hex=1:2). When the reaction was completed, H₂O was added to the reaction solution to quench the reaction, and extracted with (EA)/H₂O. The organic layer was washed with distilled water three times, and water in the organic layer was removed by using MgSO₄, and then the organic layer was concentrated. The concentrate was purified by flash column chromatography using mixed solution of (EA:Hex)=1:4 (volume ratio) to afford 1.07 g of the target compound at yield of 78.3%.

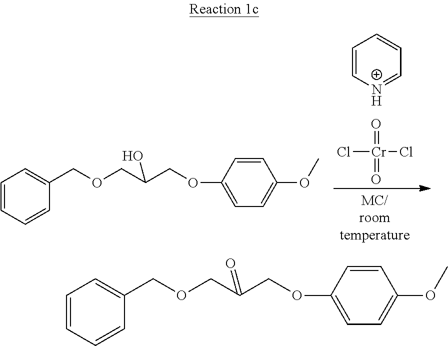

As shown in the above Reaction 1c, 233.5 ml of methylene chloride (MC) was combined with 52.77 g (29.79 mmole) of pyridinium chlorochromate (PCC) and 52.77 g of celite and the mixture was stirred. The product 23.55 (81.67 mmole) of Reaction 1b was dissolved in 81.2 ml of MC added dropwise, and was stirred at room temperature for 24 hours. The reaction was monitored by TLC (EA:Hex=1:2). When the reaction was complete, the reaction mixture was filtered using celite filter, and the filtrate was concentrated and purified using flash column (EA:MC:Hex=1:1:4 (volume ratio) mixed solution) to obtain 14.2 g of the target compound at yield of 60.7%.

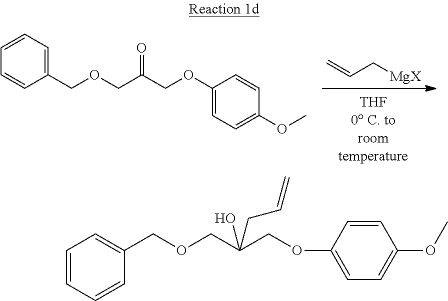

As shown in the above Reaction 1d, 370 mg (1.29 mmole) of the product (SM) of the Reaction 1c was dissolved in 1.3 ml of THF, and after bubbling with N₂, the mixture was cooled to a temperature of 0° C. 2M of allymagnesium chloride in THF 1.94 ml (3.877 mmole) was added dropwised and stirred at room temperature for 2 hours. The reaction was monitored by TLC (EA:HEX=1:4). When the reaction was completed, diluted hydrochloric acid aqueous solution was added to the reaction solution to quench the reaction, and the reaction product was extracted with (EA)/H₂O, and water was removed with MgSO₄, followed by concentration of the reaction product. The concentrate was purified by flash column chromatography (EA:Hex=1:7 (volume ratio) mixed mixture) to afford 250 mg of the target compound at yield of 59%.

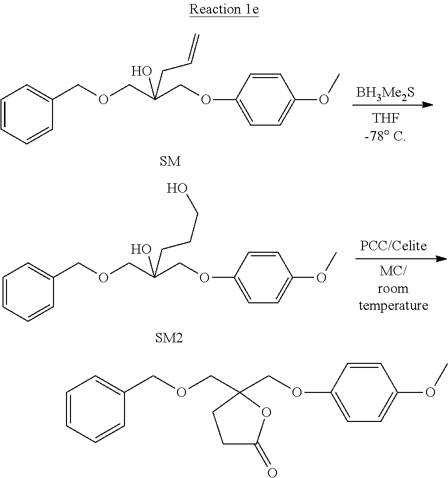

As shown in the above Reaction 1e, after dissolving 6.5 g (19.793 mmole) of the product (SM) of the Reaction 1d in 130 ml of THF and following bubbling with N₂, the reaction mixture was cooled to a temperature of −78° C., and borane dimethyl sulfide (2M borane dimethyl sulfide solution in THF, 16 ml (31.977 mmole) of BH₃Me₂S) was added, and the mixture was stirred overnight at the same temperature. The reaction was monitored by TLC (EA:Hex=1:2). When the reaction was complete, MeOH was added to the reaction solution to quench the reaction and the reaction was concentrated. 42.6 g (197.93 mmole) of pyridinium chlorochromate (PCC) and 42.6 g of celite were added to 32.5 ml MC and stirred. A solution containing the above concentrated reaction product SM2 was added to an appropriate amount of MC, and the mixture was stirred at room temperature for 5 hours. The reaction was monitored by TLC (EA:Hex=1:2). After the reaction was completed, it was filtered through celite, concentrated, and purified by flash column chromatography (EA:Hex=1:4 (volume ratio) mixture) to afford 4.56 g of the desired product at yield of 67.3%.

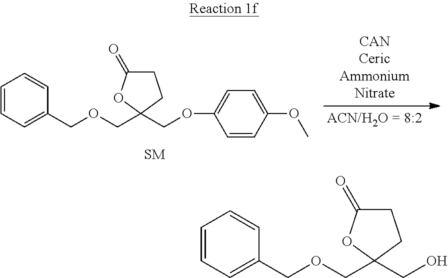 [SMILES: COc1ccc(OCC2(COCc3ccccc3)CCC(=O)O2)cc1 ; O=C1CCC(CO)(COCc2ccccc2)O1]

As shown in the above Reaction 1f; 2.94 g (8.59 mmole) of the product (SM) of Reaction 1e was dissolved in 70 ml of a mixture of Acetonitrile and ACN/H₂O=8:2 (volume ratio), and after cooling to a temperature of 0° C., 14.2 g (25.76 mmole) of ceric ammonium nitrate (CAN) was added at the same temperature. The reaction was monitored to be TLC (EA:Hex=1:2). When the reaction was complete, saturated NaHCO₃ (aq. solution) was added to the reaction solution. After quenching the reaction by adding and allowing the reaction to warm to room temperature. (EA)/H₂O extraction. After removing water with MgSO₄, the mixture was concentrated to obtain 2.8 g of the target compound at yield of 137.9%.

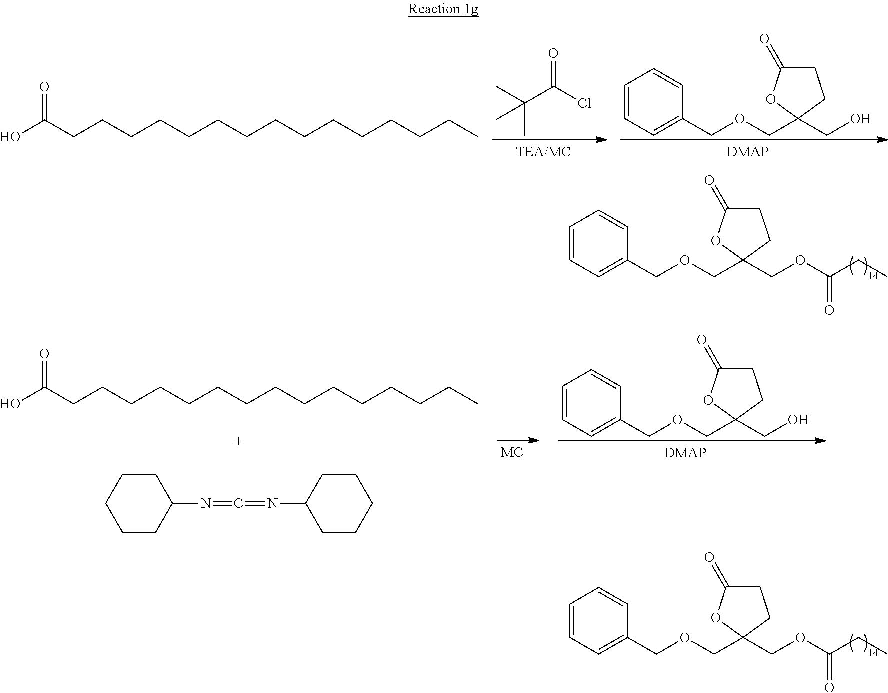

As shown in the above reaction 1 g, 58.8 ml of MC was added with 5.88 g (22.945 mmole) of palmitic acid and cooled to 0° C. While maintaining the same temperature, TEA (7.4 ml, 52.95 mmole) was slowly dropped in the solution and stirred at the same temperature for 30 minutes. 1.47 g (17.65 mmole) of the product of Reaction 1f was added, 4-dimethylamino pyridine and 216 mg (1.765 mmole) of DMAP were added, and the temperature was raised to room temperature, followed by stirring at the same temperature for 2 hours. The reaction was monitored to be TLC (EA:Hex=1:2). When the reaction was complete, the reaction produce was extracted with a solution of KOH (aq.solution)/MC, and then with HCl solution/MC, concentrate, and purified by flash column chromatography (EA:Hex=1:4.5 (volume ratio) mixture)). 1.9 g of the target compound was obtained at yield 22.6%.

As of alternative method, the product of Reaction 1f (2.8 g, 11.85 mmole, 1 eq.)/palmitic acid (1.3 eq.)/N,N1-dicyclohexylcarbodiimide (DCC) 1.3 eq./TLC (EA:Hex=1:2) were reacted. When the reaction was complete, the solvent was concentrated, and added to the appropriate amount of hexane slurried, filtered, and the reaction product was concentrated and then purified on the flash column chromatography (EA:Hex=1:7 (volume ratio) mixture) to afford 3.66 g of the target compound at yield of 65%.

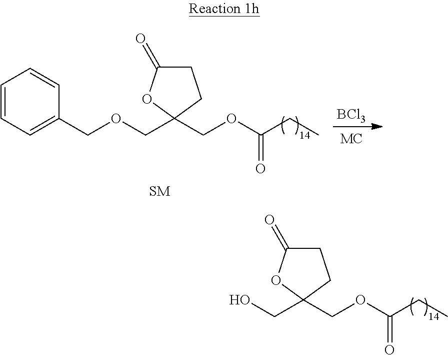

As shown in the above Reaction 1h, 3.66 g (7.71 mmole) of the product (SM) of the Reaction 1g was added into 38 ml of MC, bubbled in N2, and then cooled to a temperature of −78°. Boron trichloride (1 M in MC) and BCl₃ (23 ml, 23.11 mmole) were slowly dropped at the same temperature, and the mixture was stirred at the same temperature for 2 hours. The reaction was monitored by TLC (EA:Hex=1:1). When the reaction was completed, saturated NaHCO₃ solution was added to quench the reaction and then extracted with MC/H₂O. Then the organic layer was separated and concentrates, and finally purified by flash column chromatography with EA:Hex=1:2 (volume ratio) to afford 2.68 g of compound at yield of 90%.

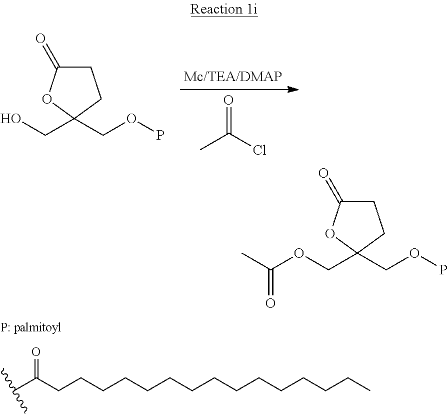

As shown in the above reaction 1i, 100 mg (0.26 mmole) of the reaction product of Reaction 1h was treated with 72.5 mL (0.52 mmole) of TEA and 3.17 mg (0.026 mmole) of the 4-dimethylamino pyridine (DMAP) in 10 ml of MC and stirred for 30 minutes at room temperature. Acetyl chloride (24 mL, 0.338 mmole) was slowly dropped in the reaction solution and stirred at the same temperature for 2 hours. The reaction was monitored by TLC (EA:Hex=1:2). When the reaction was complete, the reaction mixture was extracted with KOH (aq. solution)/MC and with HCl solution/MC, and then concentrated and purified using flash column chromatography with EA:Hex=1:3 (volume ratio). 87.4 mg of the target compound was obtained at yield of 78.8%.

### Example 2: Synthesis of EC-A51

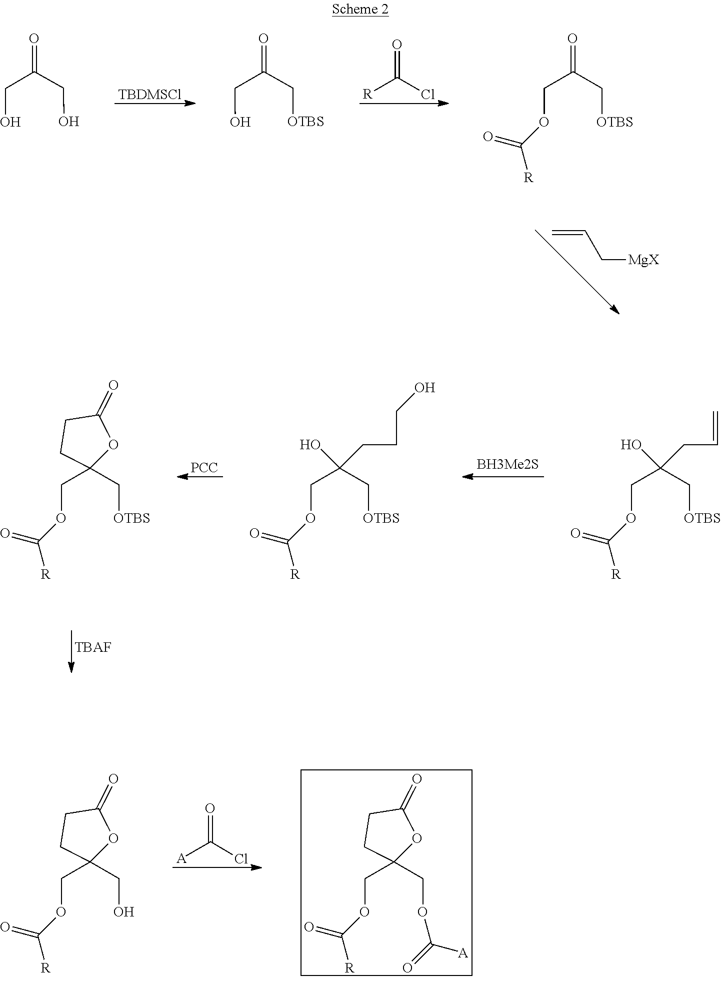

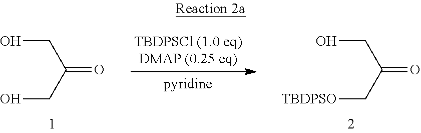

To a mixture of Compound 1 (10.00 g, 111.01 mmol, 1.00 eq), DMAP (3.53 g, 28.86 mmol, 0.26 eq) in pyridine (350.00 mL) was dropwise added TBDPSCl (30.51 g, 111.01 mmol, 28.51 mL, 1.00 eq) at a temperature of 0° C. under N₂ atmosphere. The mixture was stirred at a temperature of 0° C. for 15 min, then warmed to a temperature of 25-30° C. and stirred for 16 hours. TLC (PE:EA=10:1) showed the reaction was completed. The aqueous phase was extracted with ethyl acetate (500 ml×4). The combined organic phase was washed with 1M HCl (500 mL) then was washed with brine (2000 mL×1), dried with anhydrous Na₂SO₄, filtered and concentrated in vacuum. The residue was purified by flash column chromatography (gradient eluent of petroleum ether (PE)/ethyl acetate (EA) from 200:1 to 50:1) to afford Compound 2 (7.00 g, 21.31 mmol, 19.20% yield) (rf=0.33) as yellow oil.

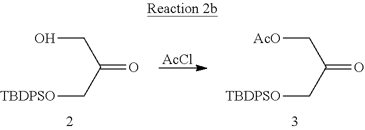

To a mixture of Compound 2 (300.00 mg, 913.30 umol, 1.00 eq), pyridine (361.21 mg, 4.57 mmol, 368.58 uL, 5.00 eq) and DMAP (16.74 mg, 136.99 umol, 0.15 eq) in DCM (10.00 mL) was added acetyl chloride (78.86 mg, 1.00 mmol, 71.69 uL, 1.10 eq) dropwise at a temperature of 0 C under N₂ atmosphere. The mixture was stirred at a temperature of 0° C. for 5 mins, then warmed to a temperature of 25-30° C. and stirred for 4 hours. TLC (PE:EA=5:1) showed the reaction was completed. The residue was poured into ice-water (w/w=1/1) (100 mL) and stirred for 5 mins. The aqueous phase was extracted with ethyl acetate (100 mL×4). The combined organic phase was washed with 1M HCl (100 mL) then was washed with brine (200 mL×1), dried with anhydrous Na₂SO₄, filtered and concentrated in vacuum. The residue was purified by flash column chromatography (gradient eluent of PE:EA from 200:1 to 50:1) to afford Compound 3 (200.00 mg, 539.80 umol, 59.10% yield) as yellow oil (rf=0.5).

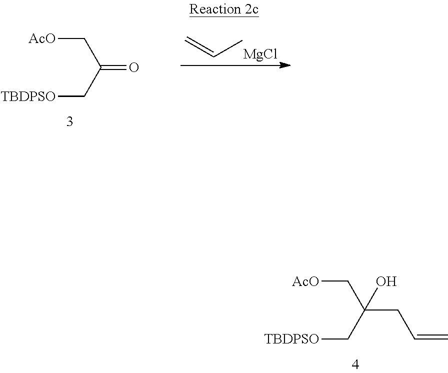

To a mixture of Compound 3 (250.00 mg, 674.75 umol, 1.00 eq) in THF (2.00 mL), allyl magnesium chloride (2 M, 337.37 uL, 1.00 eq) was added dropwise at a temperature of −70° C. under N₂ atmosphere. The mixture was stirred at a temperature of −70° C. for 15 mins, then warmed to a temperature of 25-30° C. and stirred for 3 hours. TLC (PE:EA=10:1) showed the reaction was completed. The residue was poured into ice-water (w/w=1/1) (100 mL) and stirred for 5 mins. The aqueous phase was extracted with ethyl acetate (100 mL×4). The combined organic phase was washed with 1M HCl (100 mL) then was washed with brine (200 mL×1), dried with anhydrous Na₂SO₄, filtered and concentrated in vacuum. The residue was purified by flash column chromatography (gradient eluent of PE:EA from 200:1 to 50:1) to afford Compound 4 (100.00 mg, 193.90 μmol, 28.74% yield, 80% purity) (rf=0.50) as colorless oil.

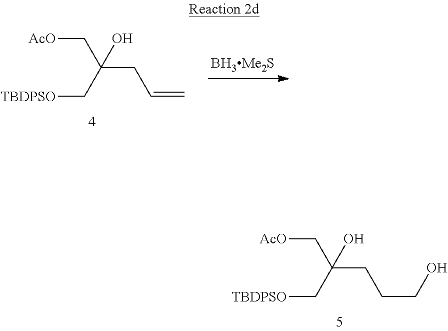

To a mixture of Compound 4 (2.00 g, 4.85 mmol, 1.00 eq) in THF (50.00 mL), BH₃-Me₂S (10 M, 970.00 uL, 2.00 eq) was added dropwise at a temperature of −78° C. under N₂ atmosphere. The mixture was stirred at a temperature of −78° C. for 30 mins, then warmed to 25-35° C. and stirred for 15.5 hours. TLC (PE:EA=10:1) showed the reaction was completed. The reaction was quenched with MeOH (80 mL). The combined organic phase concentrated in vacuum. This afforded Compound 5 (2.50 g, crude) as yellow oil, and we used it for the next step directly.

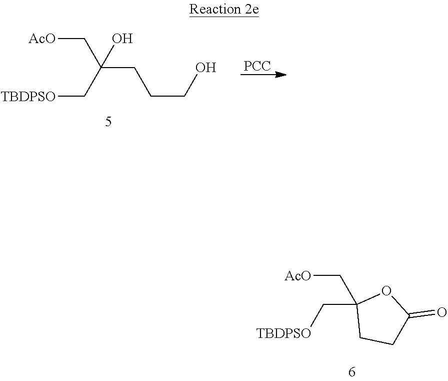

To a mixture of Compound 5 (110.00 mg, 255.45 umol, 1.00 eq) in DCM (10.00 mL), PCC (550.65 mg, 2.55 mmol, 10.00 eq) was added in one portion at a temperature of 25-30° C. under N₂. The mixture was stirred at a temperature of 25-30° C. for 36 hours. TLC (PE:EA=2:1) showed the reaction was completed, and several new spots was detected. The reaction was filtered and the filtrate was extracted with DCM (100 mL×4). Then was washed with brine (200 mL×1), dried with anhydrous Na₂SO₄, filtered and concentrated in vacuum. The residue was purified by Prep-TLC (PE:EA=5:1). This afforded Compound 6 (60.00 mg, 112.52 μmol, 44.05% yield, 80% purity) as yellow oil.

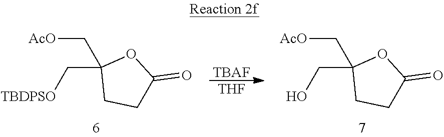

To a mixture of Compound 6 (200.00 mg, 468.85 umol, 1.00 eq) in THF (2.00 mL), TBAF (1 M, 609.51 μL, 1.30 eq) was added in one portion at a temperature of 25-35° C. under N₂ atmosphere. The mixture was stirred at a temperature of 25-35° C. for 2 hours. TLC (PE:EA=10:1) showed the reaction was completed. The reaction was filtered and concentrated in vacuum. The residue was purified by Prep-TLC (PA:EA=10:1) to afford Compound 6 (60.00 mg, 255.07 umol, 54.40% yield, 80% purity) as colorless oil.

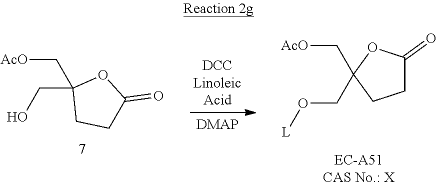

To a mixture of (9Z,12Z)-octadeca-9,12-dienoic acid (35.00 mg, 124.80 umol, 35.00 uL, 1.00 eq) and Compound 7 (19.96 mg, 106.08 umol, 0.85 eq) in DCM (2.00 mL) DCC (30.90 mg, 149.76 umol, 30.29 uL, 1.20 eq) and DMAP (3.05 mg, 24.96 umol, 0.20 eq) were added in one portion at a temperature of 25-35° C. under N₂ atmosphere. The mixture was stirred at a temperature of 25-35° C. for 16 hours. TLC (PE:EA=10:1) showed the reaction was completed. The residue was poured into ice-water (w/w=1/1) (50 mL) and stirred for 5 mins. The aqueous phase was extracted with ethyl acetate (50 mL×4). The combined organic phase was washed with brine (200 mL×1), dried with anhydrous Na₂SO₄, filtered and concentrated in vacuum. The residue was purified by Prep-TLC (PE:EA=10:1) with EW2688-135-P1 (20 mg) for 4 times to afford EC-A51 (20.00 mg, 42.17 umol, 33.79% yield, 95% purity) (rf=0.24) as colorless oil.

### Example 3: Synthesis of EC-A32

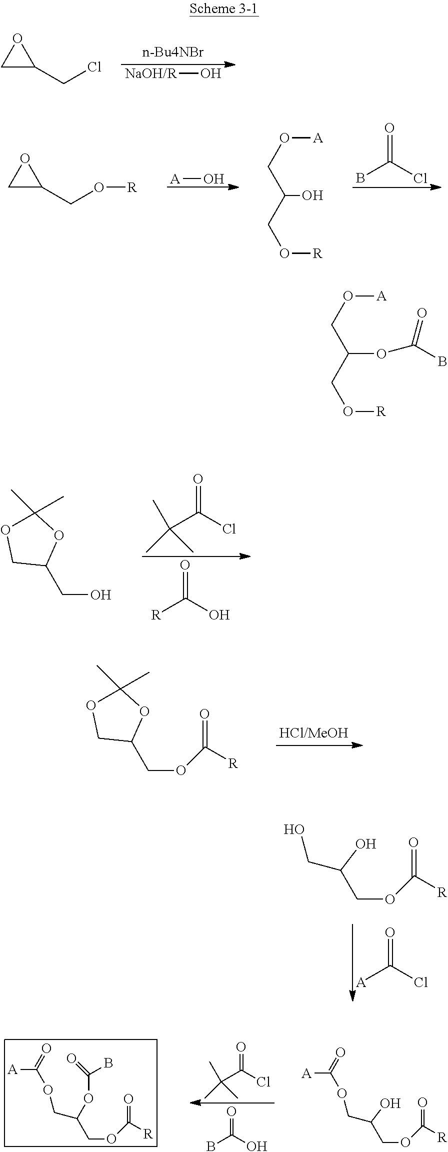

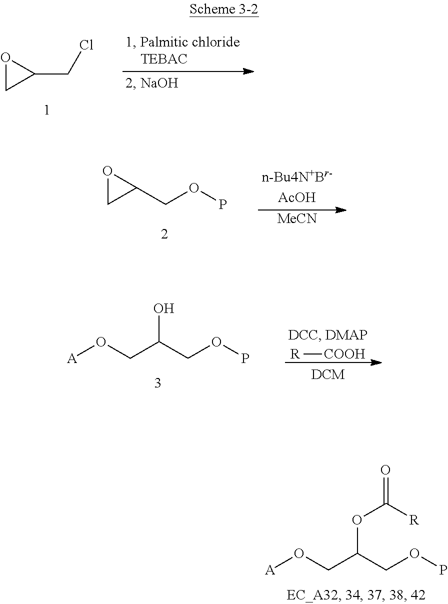

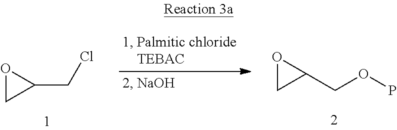

To a solution of palmitic acid (11.09 g, 43.23 mmol, 1.00 eq) in 2-(chloromethyl)oxirane (40.00 g, 432.34 mmol, 10.00 eq), TEBAC (984.74 mg, 4.32 mmol, 0.10 eq) was added, then the reaction temperature was raised to a temperature of 117° C. and the reaction continued at that temperature for 2 h. After the mixture was cooled to a temperature of 60° C., NaOH (2.08 g, 51.88 mmol, 1.20 eq) pellets was added, and the pellets soon turned into tiny particles in suspension. The mixture was stirred at a temperature of 60° C. for 3 h. The residue was purified by silica gel chromatography (gradient eluent of PE:EA from 100:1 to 10:1). Compound 2 (11.00 g, 35.20 mmol, 81.42% yield) was obtained as a white solid.

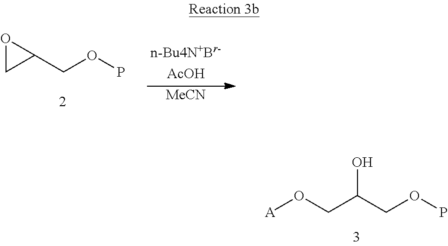

To a solution of oxiran-2-ylmethyl hexadecanoate (2.00 g, 6.40 mmol, 1.00 eq) in MeCN (20.00 mL), acetic acid (307.47 mg, 5.12 mmol, 0.80 eq) and cat. amount of Bu4NBr (206.32 mg, 640.00 umol, 0.10 eq) were added and the solution was stirred at a temperature of 100° C. for 16 h. The reaction mixture was concentrated under reduced pressure to remove solvent. The residue was purified by column chromatography (PE:EA=50:1-5:1). Compound 3 (1.00 g, crude) was obtained as a white solid and the crude product was used in the next step directly.

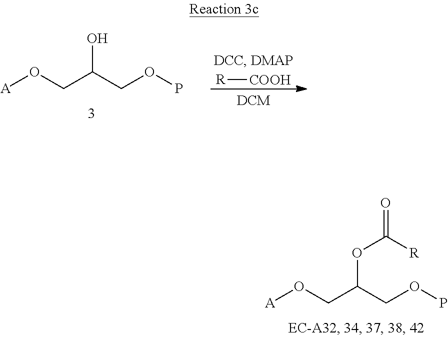

To a solution of (Z)-hexadec-9-enoic acid (56.91 mg, 223.69 umol, 1.00 eq) in DCM (1.00 mL) (3-acetoxy-2-hydroxy-propyl) hexadecanoate (100.00 mg, 268.43 umol, 1.20 eq), DCC (55.38 mg, 268.43 umol, 1.2 eq) and cat. amount of DMAP (5.47 mg, 44.74 umol, 0.20 eq) were added, and the solution was stirred at a temperature of 25° C. for 18 h. The reaction mixture was concentrated under reduced pressure to remove solvent. The residue was purified by prep-TLC. Compound EC-A32 (60.00 mg, 96.56 umol, 43.17% yield, 98% purity) (Rf=0.70) was obtained as a white solid.

### Example 4

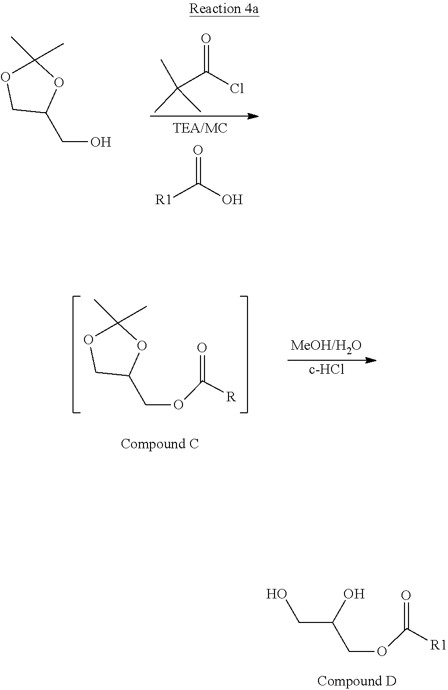

As shown in the above Reaction 4a, after 2.09 eq. of triethylamine (TEA) and 1.0 eq. of R1-OH (R1=palmitoyl) were dissolved in 33 ml of methylenechloride (MC), the reaction temperature was cooled to 5-15° C., then 1.05 eq. of pivaloyl chloride was added and stirred by maintaining the reaction temperature at or less than 15° C. for 30 minutes. To the reaction mixture above, 2.54 ml (20.47 nmole, 1.05 eq.) of solketal was added rapidly and subsequently 0.01 eq. of DMAP was added. The reaction mixture was stirred at a temperature of 20-25° C. for 1 hour. When the reaction was completed, 12.5 ml of distilled water was added to the reaction solution to separate the layers, additional 12.5 ml of distilled water was added, and then pH was adjusted to pH 7-8 by adding 0.3 ml of c-HCl. The organic layer was separated and concentrated to obtain a compound in oil phase. To this compound, 15 ml of methanol, 1.75 ml of distilled water were added, the temperature was set at 22-23° C., and then 2 ml of c-HCl was added slowly dropwise. The reaction solution was stirred under the temperature of 25° C. for 2-2.5 hours, and white solid was precipitated. To this solid precipitate, 13 ml of hexane and 16 ml of distilled water were added, as maintaining the temperature at 25° C., 1.9 ml of pyridine was added and pH was adjusted at pH 4-5. Then the temperature was cooled to 15° C. and the reaction solution was filtered. The filtered compound was washed with hexane and dried to obtain the compound D (R1 is palmitoyl) at yield of 85%.

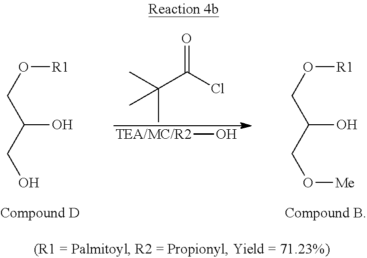

As shown in Reaction 2b above, 8.53 ml of pyridine, 5 g of compound D obtained in Reaction 2a (R1=Palmitoyl, 15.13 mmol, 1 eq.) and DMAP (0.02 eq.) were added to 30 ml of MC, and then dissolute at the temperature of 25 to 30° C. and after that the temperature was cooled to 20° C., and propionyl chloride (0.2 eq) was slowly added dropwise. The reaction temperature was cooled to 18 or 19° C., propionyl chloride (0.3 eq) dissolved in MC was added dropwise, and the temperature was cooled to a temperature of 13 or 15° C. Propionyl chloride (0.5 eq) was added dropwise, the temperature was again cooled to 5 to 10° C., and propionyl chloride (0.5 eq) was added dropwise, followed by stirring for 1 hour. 20 ml of purified water was added at the same temperature, and 6 ml of c-HCl was added to adjust the pH to 1-2. The organic layer was thoroughly neutralized with K₂CO₃ and MgSO₄. dried and concentrated. Concentrated with hexane to remove residual MC. After adding 15 ml of hexane, the temperature was cooled to 18 to 20° C., and seeding was performed to precipitate crystals. After depositing the reaction product at a temperature of 13 to 15° C., it was cooled to 10° C. again, washed with cooled hexane, and dried to obtain the target compound B at yield 71.23%. Using the obtained compound B (1 eq. R1=Palmitoyl, R2=Propionyl), along with the Reaction 1c of EXAMPLE 1, the target compound represented by the chemical formula 2 (EC-A78, R1=Palmitoyl, R2=Propionyl, R3=Linoleoyl) was obtained.

### Example 5: Synthesis of EC-A04

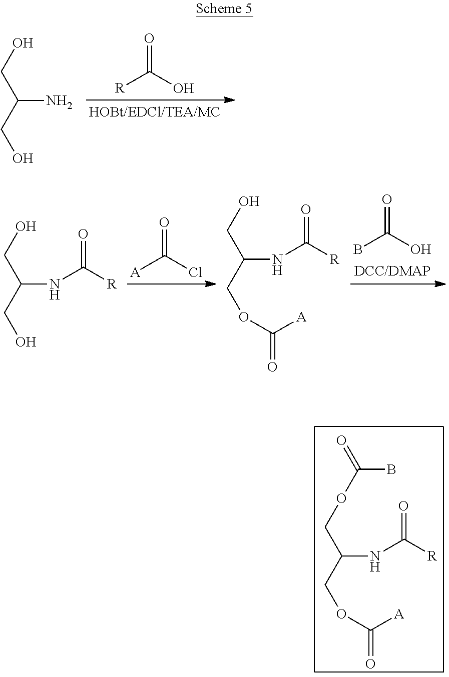

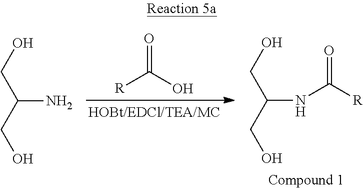

2-Amino propane-1,3-diol, 1.5 eq TEA, linoleic acid, 2 g, 7.13 mmole, 1 eq.), HOBt (1-Hydroxybenzotriazole, 1.2 eq.) and EDCI (N-3 Dimethylamino propyl)-(N-ethylcarbodiimide, 0.2 eq.), as a starting material, were combined in 500 ml of MC and stirred at 25° C. for 18 hours. The reaction solution was concentrated and purified by column (gradient eluent MC: MeOH from 100:1 to 10:1) to obtain the compound 1 (L=Linolcoyl). (MeOH=methanol, Yield 90.46%).

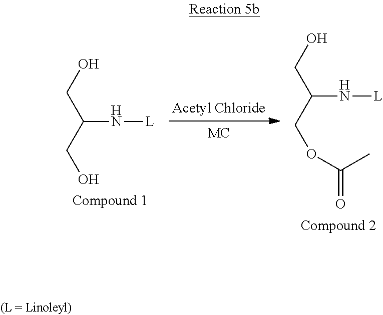

The compound 1 (1 g, 2.83 mmole, 1 eq.) synthesized in Reaction 5a was added to 10 ml of MC and dissolved, and then acetyl chloride (0.8 eq) was slowly added dropwise, while maintaining the temperature of 0° C. The reaction solution was concentrated and purified by column (gradient eluent MC:MeOH from 10:1 to 1:1) to afford the compound 2 at yield 74.25%.

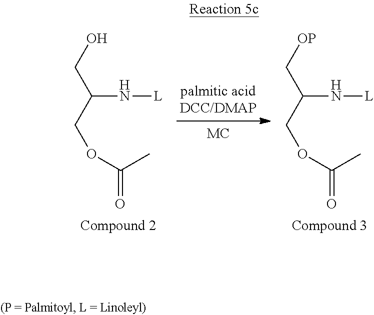

The compound 2 (100 mg. 252.8 mmole. 1 eq.) synthesized in Reaction 5b, DCC (N,N1-Dicyclohexylcarbodiimide, 1.2 eq.) and DMAP (4-Dimethylamino pyridine, 0.2 eq.) were combined in 100 ml of MC and stirred at 25° C. for 18 hours. The reaction solution was concentrated and purified with a column (gradient eluent PE:EA from 30:1 to 10:1) to obtain the compound 3, EC-A04, (P=Palmitoyl, L=Linoleoyl) at yield of 60.4%.

### Example 6: Synthesis of EC-A06

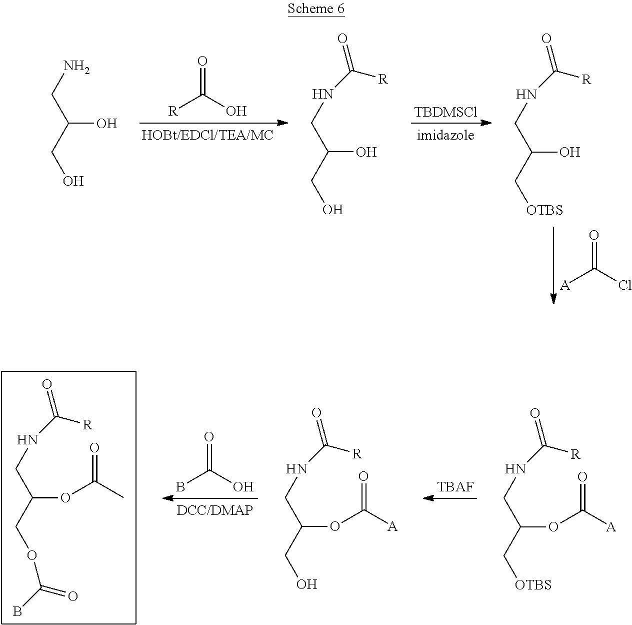

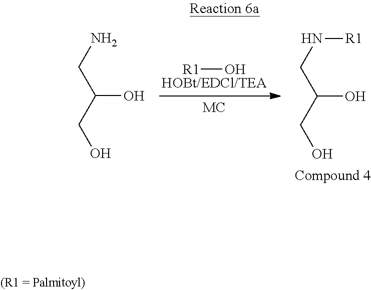

3-Amino-1,2-propanediol, 1.2 eq R1-OH (g, 3.9 mmol, 1 eq.), EDCI (N-3 Dimethylamino propyl)-N′dethylcarbodiimide, 1.2 eq.), HOB (1-Hydroxybenzotriazole, 1.2 eq.) and TEA (6 eq.), as a starting material, were added in 360 ml of MC and the reaction mixture was stirred at 20° C. for 16 hours. The reaction was monitored by TLC (MC:MeOH=10:1) and the starting material was completely consumed. The reaction solution was concentrated and purified by column (gradient eluent MC:MeOH from 20:1 to 10:1) to obtain the compound 4 (R1=Palmitoyl) at yield of 53.22%.

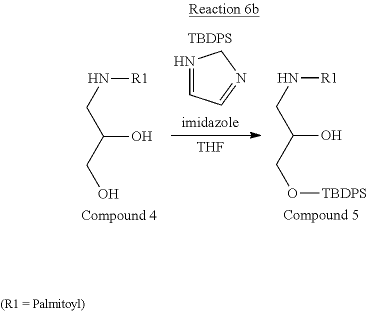

The compound 4 (720 mg, 2.18 mmole, 1 eq.) obtained in the above Reaction 6a was added to 10 ml of THF (Tetrahydrofuran), and TBDPSCI (tert-Butyldiphenylchlorosilance, 1.2 eq.) and imidazole (2 eq.) were further added to the mixture. The reaction mixture was stirred at a temperature of 20° C. for 16 hours. The reaction was monitored by TLC (MC:MeO=10:1, Rf=0.7) and the starting material was completely consumed. The reaction solution was concentrated and purified by column to afford the compound 5 (R1=Palmitoyl, TBDPS=tert-butyldiphenylchlorosilance) at yield of 76.73%.

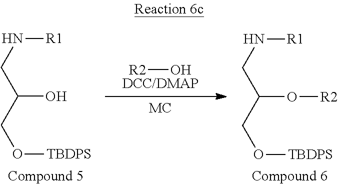

The compound 5 (500 mg, 880.41 mmole, 1 eq.) synthesized in the above Reaction 6b, R2-OH (1.05 eq.), and DCC (1.05 eq) were added to 1 ml of MC, and the mixture was stirred at a temperature of 20 to 25° C. for 16 hours. The reaction was monitored by TLC (PE:EA=3:1, Rf=035) and the starting material was completely consumed. The reaction solution was concentrated and purified by column (gradient eluent PE:EA from 3:1 to 1:1) to afford the target compound 6 (R1=Palmitoyl, R2=Linoleoyl) at yield of 64.98%.

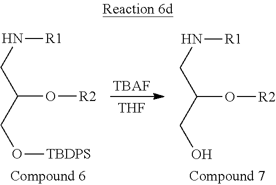

The compound 6 (500 mg, 602.16 mmole, 1 eq.) synthesized in the above Reaction 6c and TBAF (Tetrabutylammonium fluoride hydrate, 1.5 eq.) were added to 6 ml of THF, and the mixture was stirred at a temperature of 20 to 25° C. for 16 hours. The reaction was monitored by TLC (PE:EA=3:1, R_{f}=0.1) and the starting material was completely consumed. The reaction solution was concentrated and purified by column (gradient eluent PE:EA from 3:1 to 1:1) to afford the target compound 7 (R1=Palmitoyl, R2=Linolcoyl) at yield of 90.34%.

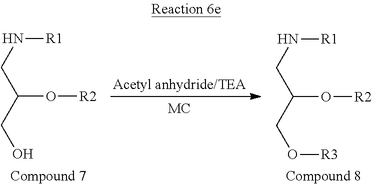

Compound 7 (100 mg, 168.93 mmole, 1 eq.) synthesized by the above Reaction 6d and acetic anhydride (1.2 eq.) and TEA (2 eq.) were combined in 1 mL MC and stirred at a temperature of 20-25° C. for 16 hours. The reaction was monitored by TLC (PE:EA=3:1, Rf=0.35) and the starting material was completely consumed. The reaction solution was concentrated, and purified by column (PE:EA=3:1) to obtain the target compound 8 (EC_A06, R1=Palmitoyl, R2=Linoleoyl, R3=Acetyl) at yield of 19.47%.

### Example 7: Synthesis of Glycerol Derivative

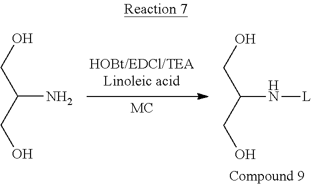

2-Amino propane-1,3-diol (1.5 eq.) as starting material, Triethylamine (TEA, 6 eq.), linoleic acid (2 g, 7.13 mmole, 1 eq.), HOBt (1-Hydroxybenzotriazole, 1.2 eq.) and EDCI (N-(3-Dimethylamino propyl)-N′-ethylcarbodiimide, 1.2 eq.) were added to 500 ml of MC (Methylene chloride), and stirred at 25° C. for 18 hours. The solvent was concentrated and purified by column (MC:MeOH=100:1.fwdarw.10:1) to obtain the target compound 9 (L=linoleoyl, MeOH=methanol, yield 90.46%).

### Example 8: Synthesis of Glycerol Derivative

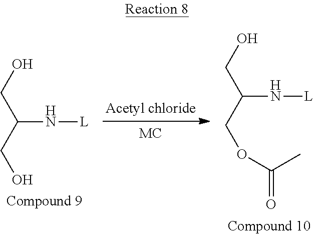

Compound 9 synthesized in Example 7 (1 g, 2.83 mmole, 1 eq.) was added and dissolved to 10 ml of MC (Methylene chloride). And acetyl chloride (0.8 eq.) was slowly added dropwise while maintaining 0° C. The reaction solution was stirred at 25° C. for 18 hours. The solvent was concentrated and purified by column (MC:MeOH=10:1.fwdarw.1:1) to obtain the target compound 10 (yield 74.25%).

### Example 9: Synthesis of Glycerol Derivative

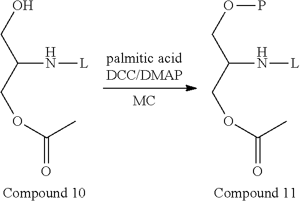

Compound 10 synthesized in Example 8 (100 mg, 252.8 mmole, 1 eq.), DCC(N,N′-Dicyclohexylcarbodiimide, 1.2 eq.) and DMAP (4-(Dimethylamino) pyridine, 0.2 eq.) were added to 100 ml of MC (Methylene chloride), and stirred at 25° C. for 18 hours. The solvent was concentrated and purified by column (PE (Petroleum ether):EA (Ethyl acetate)=30:1.fwdarw.10:1) to obtain the target compound 11 (P=palmitoyl, L=linoleoyl, yield 60.4%).

### Example 10: Synthesis of Glycerol Derivative

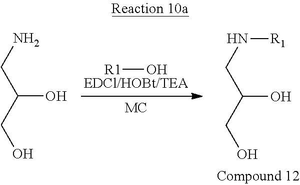

3-Amino-1,2-propane diol (1.2 eq.) as starting material, R₁—OH (1 g, 3.9 mmol, 1 eq.), EDCI (N-(3-Dimethylamino propyl)-N′-dethylcarbodiimide, 1.2 eq.), HOBt (1-Hydroxybenzotriazole, 1.2 eq.) and TEA (6 eq.) were added to 360 ml of MC (Methylene chloride), and stirred at 20° C. for 16 hours. The reaction was confirmed by TLC (MC:MeOH=10:1) (TLC=Thin Layer Chromatography). SM was completely consumed. The solvent was concentrated and purified by column (MC:MeOH=20:1.fwdarw.10:1) to obtain the target compound 12 (R.sub.1=palmitoyl, yield 53.22%).

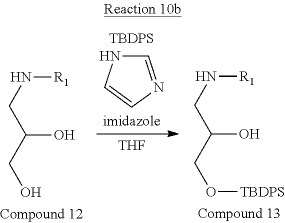

Compound 12 synthesized in Reaction 10a (720 mg, 2.18 mmole, 1 eq.) was added to 10 ml of THE (Tetrahydrofuran), and TBDPSCI (tert-Butyldiphenylchlorosilane, 1.2 eq.) and imidazole (2 eq.) were added, and stirred at 20° C. for 16 hours. The reaction was confirmed by TLC (MC:MeOH=10:1, Rf=0.7). SM was completely consumed. The reaction solution was concentrated and purified by column to obtain the target compound 5 (R¹=palmitoyl, TBDPS=tert-butyldiphenylsilyl, yield=76.73%).

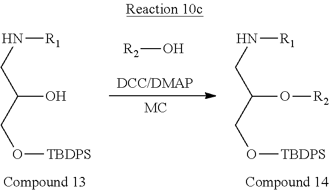

Compound 13 synthesized in Reaction 10b (500 mg, 880.41 mmole, 1 eq.), R₂—OH (1.05 eq.), DCC (1.05 eq.) and DMAP (0.1 eq.) were added to 1 ml of MC, and stirred at 20.about.25° C. for 16 hours. The reaction was confirmed by TLC (PE:EA=3:1, Rf=0.35). SM was completely consumed. The reaction solution was concentrated and purified by column (PE:EA=3:1.fwdarw.1:1) to obtain the target compound 14 (R₁=palmitoyl, R2=linoleoyl, yield=64.98%).

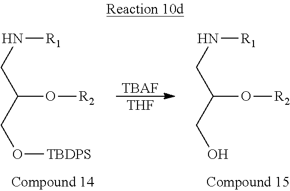

Compound 14 synthesized in Reaction 10c (500 mg, 602.16 mmole, 1 eq.) and TBAF (Tetrabutylammonium fluoride hydrate, 1.5 eq.) were added to 6 ml of THF and stirred at 20.about.25° C. for 16 hours. The reaction was confirmed by TLC (PE:EA=3:1, Rf=0.1). SM was completely consumed. The reaction solution was concentrated and purified by column (PE:EA=3:1.fwdarw.1:1) to obtain the target compound 15 (R1=palmitoyl, R2=linoleoyl, yield=90.34%).

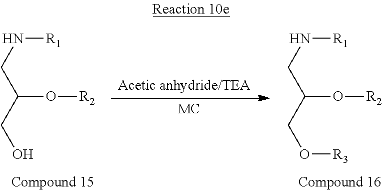

Compound 15 synthesized in Reaction 10d (500 mg, 602.16 mmole, 1 eq.), acetic anhydride (1.2 eq.) and TEA (2 eq.) were added to 1 ml of MC and stirred at 20.about.25° C. for 16 hours. The reaction was confirmed by TLC (PE:EA=3:1, Rf=0.35). SM was completely consumed. The reaction solution was concentrated and purified by column (PE:EA=3:1) to obtain the target compound 16 (EC_A06, R.sub.1=palmitoyl, R2=linoleoyl, R3=acetyl, yield=19.47%).

### Example 11: Synthesis of Glycerol Derivative

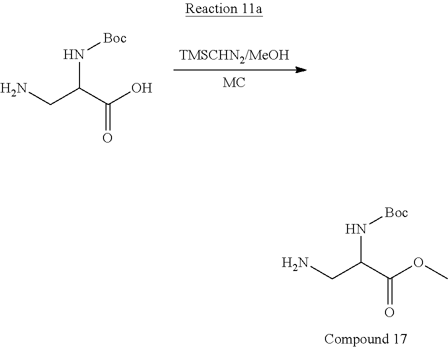

2-[(tert-Butoxycarbonyl)amino]-3-aminopropionic acid (Boc-Dap-OH, 10 g, 48.97 mole, 1 eq.) as starting material, and MeOH (16 ml) were added to 160 ml of MC, and (Trimethylsilyl) diazomethane (TMSCHN.sub.2, (Trimethylsilyl) diazomethane solution, 2.0 M in Hex. or diethyl ether, 1.07 eq.) was slowly added dropwise, and stirred at 20.about.25° C. for 16 hours. The reaction was confirmed by TLC (MC:MeOH=10:1, Rf=0.5). SM was completely consumed. The reaction solution was filtered to obtain the target compound 17.

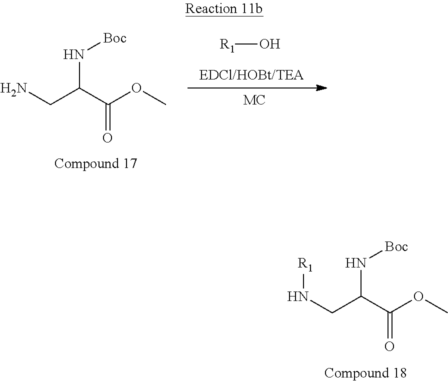

Compound 17 synthesized in Reaction 11a (1.2 eq.), R₁.about.OH (9.5 g, 37.05 mmole, 1 eq.), EDCI (1.2 eq.), HOBt (1.2 eq.) and TEA (6 eq.) were added to 100 ml of MC in the N.sub.2 purge, and stirred at 20-25° C. for 16 hours. The reaction was confirmed by TLC (PE:EA=2:1, Rf=0.5). SM was completely consumed. The reaction solution was concentrated and purified by column (PE:EA=5:1.fwdarw.3:1) to obtain the target compound 18 (R₁=palmitoyl, yield=32%).

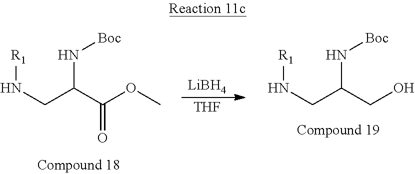

Compound 18 synthesized in Reaction 11b (2.1 g, 4.6 mmole, 1 eq.) was added to 20 ml of THF, and LiBH.sub.4 (4 eq.) was added at 0° C. and stirred at 0.about.20° C. for 1 hour. The reaction was confirmed by TLC (PE:EA=2:1, Rf=0.15). SM was completely consumed. Purified water and ethyl acetate (EA) were added to the reaction solution and extracted three times. The organic layer was washed back with brine solution (Brine soln.), dehydrated with sodium sulfate (Na.sub.2SO.sub.4), filtered, and concentrated to obtain the target compound 19 (R₁=palmitoyl, yield=91.54%).

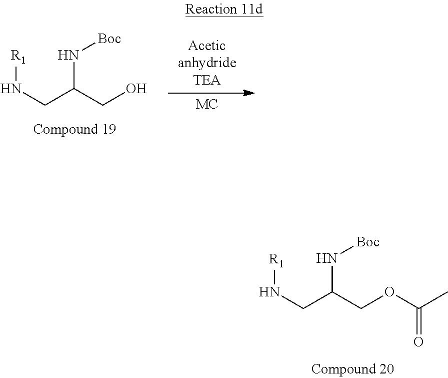

Compound 19 synthesized in Reaction 11c (200 mg, 466.58 mmole, 1 eq.) was added to 2 ml of MC. And after adding acetic anhydride (1.2 eq.) and TEA (2 eq.) at 0° C., the mixture was stirred at 0.about.20° C. for 16 hours. The reaction was confirmed by TLC (PE:EA=2:1, Rf=0.4). SM was completely consumed. Purified water and MC were added to the reaction solution and extracted three times. The organic layer was washed back with brine solution (Brine soln.), dehydrated with sodium sulfate (Na.sub.2SO.sub.4), filtered, and concentrated to obtain the target compound 20 (R₁=palmitoyl, yield=86.06%).

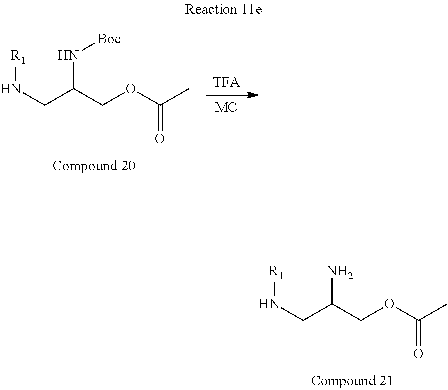

Compound 20 synthesized in Reaction 11d (230 mg, 488.65 mmol, 1 eq.) was added to a mixed solvent of 2 ml of MC and 400 ml of TFA. And it was stirred at 20° C. for 15 hours. The reaction was confirmed by TLC (MC:MeOH=10:1, Rf=0.3). SM was completely consumed. After adjusting the pH to 7-8 with sodium hydrogen carbonate (NaHCO.sub.3 soln.) in the reaction solution, and purified water and MC were added. And it was extracted three times. The organic layer was washed back with brine solution (Brine soln.), dehydrated with sodium sulfate (Na₂SO₄), filtered, and concentrated to obtain the target compound 21.

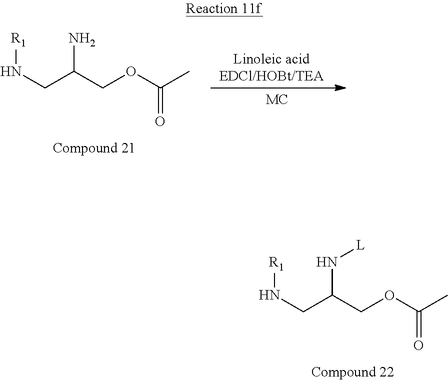

Compound 21 synthesized in Reaction 11e (181 mg, 488.44 mmol, 1 eq.), Linoleic acid (1.2 eq.), EDCI (1.2 eq.), HOBt (1.2 eq.) and TEA (4 eq.) were added to 100 ml of MC in the N.sub.2-purge. And it was stirred at 20.about.25° C. for 16 hours. The reaction was confirmed by TLC (MC:MeOH=10:1, Rf=0.7). SM was completely consumed. The reaction solution was concentrated and purified by column (PE:EA=1:1.fwdarw.3:1) to obtain the target compound 22 (EC-A44, R₁=palmitoyl, yield=9.27%).

### Example 12: Synthesis of Glycerol Derivative

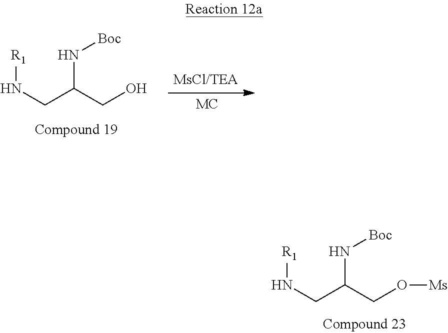

Compound 19 synthesized in Reaction 11c (500 mg, 1.17 mmol, 1 eq.) and TEA (1.1 eq.) were added to 10 ml of MC and cooled to 0° C. And then, methanesulfonyl chloride (1.1 eq.) was slowly added dropwise and stirred at 20° C. for 24 hours. The reaction was confirmed by TLC (PE:EA=2:1, Rf=0.35). Purified water and MC were added to the reaction solution and extracted three times. The organic layer was washed back with brine solution (Brine soln.), dehydrated with sodium sulfate (Na.sub.2SO.sub.4) and filtered. And it was purified by a column (PE:EA=1:1) and concentrated to obtain the target compound 23 (R₁=palmitoyl, yield=32.05%).

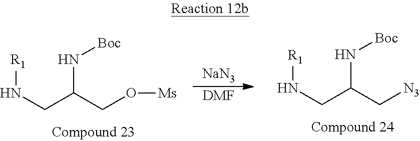

Compound 23 synthesized in Reaction 12a (300 mg, 592.02 mmol, 1 eq.) and sodium azide (2.4 eq.) were added to 6 ml of DMF (dimethylformamide) and stirred at 50° C. for 24 hours. The reaction was confirmed by TLC (PE:EA=2:1, Rf=0.35). SM was completely consumed. After adjusting the pH of 9 or more with purified water and sodium hydrogen carbonate (NaHCO.sub.3 soln.) to the reaction solution, added EA to extract three times. The organic layer was washed back with brine solution (Brine soln.), dehydrated with sodium sulfate (Na.sub.2SO.sub.4) and filtered. And it was purified by a column (PE:EA=1:1) and concentrated to obtain the target compound 24 (R₁=palmitoyl).

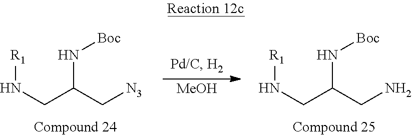

Compound 24 synthesized in Reaction 12b (300 mg, 661.29 mmol, 1 eq.) and Pd/C (300 mg) were added to 10 ml of MeOH in the N.sub.2-purge. And then, it was degassed several times with H.sub.2, and stirred for 16 hours while maintaining 20 psi of H.sub.2 at 20° C. The reaction was confirmed by TLC (MC:MeOH=10:1, Rf=0.25). SM was completely consumed. The reaction was confirmed by LC-MS (EW2692-141-P1A). When the reaction was complete, it was removed by filtration and concentrated to obtain the target compound 25 (R₁=palmitoyl).

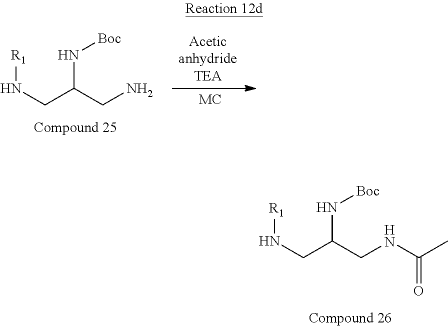

Compound 25 synthesized in Reaction 12c (3000 mg, 701.49 mmole, 1 eq.) was added to 3 ml of MC. And after adding acetic anhydride (1.2 eq.) and TEA (2 eq.) at 0° C., the mixture was stirred at 20° C. for 16 hours. The reaction was confirmed by TLC (MC:MeOH=20:1, Rf=0.3). SM was completely consumed. Purified water and MC were added to the reaction solution and extracted three times. The organic layer was washed back with brine solution (Brine soln.), dehydrated with sodium sulfate (Na.sub.2SO.sub.4) and filtered. And it was purified by a column (MC:MeOH=20:1) and concentrated to obtain the target compound 26 (R1=palmitoyl).

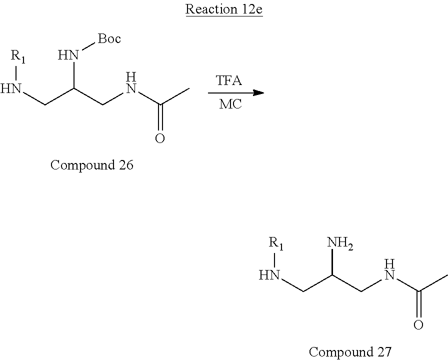

Compound 26 synthesized in Reaction 12d (50 mg, 106.45 mmol, 1 eq.) was added to a mixed solvent of 2 ml of MC and 200 ml of trifluoroacetic acid (TFA) and stirred at 20° C. for 10 hours. The reaction was confirmed by TLC (MC:MeOH=10:1, Rf=0.3). SM was completely consumed. It was adjusted the pH to 7-8 with sodium hydrogen carbonate (NaHCO.sub.3 soln.) to the reaction solution and added purified water and MC and extracted three times. The organic layer was washed back with brine solution (Brine soln.), dehydrated with sodium sulfate (Na₂SO₄), filtered, and concentrated to obtain the target compound.

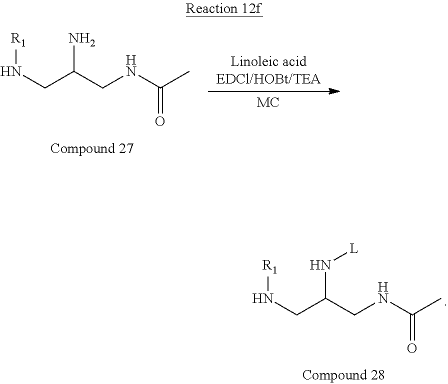

Compound 27 synthesized in Reaction 12e (40 mg, 108.23 mmol, 1 eq.), Linoleic acid (1.2 eq.), EDCI (1.2 eq.), HOBt (1.2 eq.) and TEA (4 eq.) were added to 1 ml of MC in the N.sub.2-purge, and stirred at 20.about.25° C. for 16 hours. The reaction was confirmed by TLC (MC:MeOH=10:1, Rf=0.5). SM was completely consumed. The reaction solution was concentrated and purified by column (MC:MeOH=15:1) to obtain the target compound 28 (EC-A45, R₁=palmitoyl, yield=14.16%).

Additionally, compounds disclosed herein may be synthesized using methods as described in WO2019/208980, WO2019/190137, and WO2019/177314, which are incorporated herein by reference.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A compound having a structure of the following Formula (I): 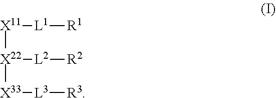
wherein:
X¹¹ is —CR^{1a}R^{1b}—, C(O)— or —NR^{1c}—;
X²² is —CR^{2a}— or —N—;
X³³ is —CR^{3a}R^{3b}—, —C(O)— or —NR^{3c}—;
L¹ is a bond, —C(O)—, —C(O)O—, —OC(O)—, —C(O)S—, SC(O)—, —NR¹¹C(O)—, —C(O)NR¹¹—, —NR¹¹C(O)NR¹²—, —NR¹¹—, —O—, —S—, —S(O)₂—, —NR¹¹S(O)₂—, —S(O)₂NR¹¹—, —NR¹¹C(O)O—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L² is a bond, —C(O)—, —C(O)O—, —OC(O)—, —C(O)S—, SC(O)—, —NR¹³C(O)—, —C(O)NR¹⁴—, —NR¹³C(O)NR¹⁴—, —NR¹³—, —O—, —S—, —S(O)₂—, —NR¹³S(O)₂—, —S(O)₂NR¹³—, —NR¹³C(O)O—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L³ is a bond, —C(O)—, —C(O)O—, —OC(O)—, —C(O)S—, SC(O)—, —NR¹⁵C(O)—, —C(O)NR¹⁵—, —NR¹⁵C(O)NR¹⁶—, —NR¹⁵—, —O—, —S—, —S(O)₂—, —NR¹⁵S(O)₂—, —S(O)₂NR¹⁵—, —NR¹⁵C(O)O—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
R¹ is —CX¹₃, —CHX¹₂, —CH₂X¹, —OCX¹₃, —OCH₂X¹, —OCHX¹₂, —N₃, —CN, —SO₂R^{1D}, —SO₂NR^{1A}R^{1B}, —NHC(O)NR^{1A}R^{1B}, —NO₂, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R² is hydrogen, halogen, —CX²₃, —CHX²₂, —CH₂X², —OCX²₃, —OCH₂X², —OCHX²₂, —N₃, —CN, —SO₂R^{2D}, —SO₂NR^{2A}R^{2B}, —NHC(O)NR^{2A}R^{2B}, —NO₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; or R^{2a} and R² together with atoms attached thereto are optionally joined to form a substituted or unsubstituted cycloalkyl, or substituted or unsubstituted heterocycloalkyl;
R³ is hydrogen, halogen, —CX³₃, —CHX³₂, —CH₂X³, —OCX³₃, —OCH₂X³, —OCHX³₂, —N₃, —CN, —SO₂R^{3D}, —SO₂NR^{3A}R^{3B}, —NHC(O)NR^{3A}R^{3B}, —NO₂, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
wherein R¹¹, R¹², R¹³, R¹⁴, R¹⁵, R¹⁶, R^{1a}, R^{1b}, R^{1c}, R^{2a}, R^{3a}, R^{3b}, R^{3c}, R^{1A}, R^{1B}, R^{1C}, R^{1D}, R^{2A}, R^{2B}, R^{2C}, R^{2D}, R^{3A}, R^{3B}, R^{3C}, and R^{3D} are independently hydrogen, —CX₃, —CHX₂, —CH₂X, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
X, X¹, X², and X³ are independently —F, —Cl, —Br, or —I,
i) with proviso that when X¹¹, X²², X³³ are —CH₂—; L¹, L², and L³ are —OC(O)—; R¹ is C₁₅ unsubstituted alkyl; and R³ is methyl, then R² is not 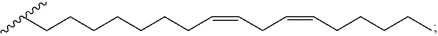 [SMILES: *CCCCCCC/C=C\C/C=C\CCCCC]
ii) with proviso that when X¹¹ and X³³ are —CH₂—, X²² is —CR^{2a}—; L¹, L², and L³ are —OC(O)—; R^{2a} and R² together with atoms attached thereto are joined to form a 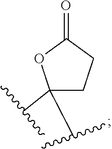 [SMILES: *C1(*)CCC(=O)O1]
and R¹ is C₁-C₆, C₈, C₁₁, C₁₃, C₁₅, C₁₉ unsubstituted alkyl, cyclopropyl, or cyclohexyl, then R³ is not 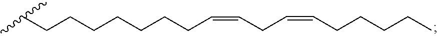 [SMILES: *CCCCCCC/C=C\C/C=C\CCCCC]
iii) with proviso that when X¹¹ and X³³ are —CH₂—, X²² is —CR^{2a}—; L¹, L², and L³ are —OC(O)—; R^{2a} and R² together with atoms attached thereto are joined to form a 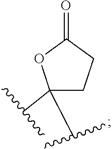 [SMILES: *C1(*)CCC(=O)O1]
and R¹ is methyl, C₃-C₄, C₁₁, C₁₃ unsubstituted alkyl, cyclopropyl, or cyclohexyl, then R³ is not unsubstituted C₁₅ alkyl;
iv) with proviso that when X¹¹, X²², X³³ are —CH₂—; L¹, L², and L³ are —OC(O)—; R¹ is C₁₅ unsubstituted alkyl; and R³ is unsubstituted C₁-C₄ alkyl, C₆, C₇, phenyl, cyclopropyl, cyclohexyl, or —CH₂—NH₂, then R² is not 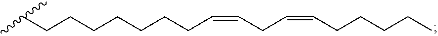 [SMILES: *CCCCCCC/C=C\C/C=C\CCCCC]
v) with proviso that when X¹¹, X²², X³³ are —CH₂—; L¹ and L² are —OC(O)—; L³ is —O—; R³ is unsubstituted C₁-C₃ alkyl, or —CH(CH₃)—OCH₃; R¹ is C₇, C₉, C₁₁ unsubstituted alkyl; and R³ is C₂-C₄, then R² is not 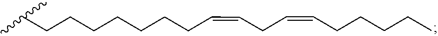 [SMILES: *CCCCCCC/C=C\C/C=C\CCCCC]
vi) with proviso that when X¹¹, X²², X³³ are —CH₂—; L¹ and L² is —OC(O)—; and L³ is —NHC(O)— or —SC(O)—; and R³ is methyl, then R² is not 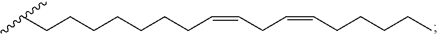 [SMILES: *CCCCCCC/C=C\C/C=C\CCCCC]
vii) with proviso that when X¹¹, X²², X³³ are —CH₂—; L¹ and L² is —OC(O)—; and L³ is —O—; and R³ is methyl or ethyl, then R² is not 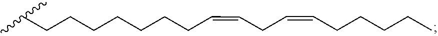 [SMILES: *CCCCCCC/C=C\C/C=C\CCCCC]
viii) with proviso that when X¹¹, X²², X³³ are —CH₂—; L¹ is —NHC(O)—; L² and L³ is —OC(O)—, R¹ is C₁₅ unsubstituted alkyl; R² is C₁-C₄ alkyl, cyclopropyl, or cyclohexyl, then R³ is not 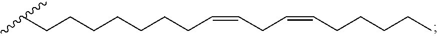 [SMILES: *CCCCCCC/C=C\C/C=C\CCCCC]
ix) with proviso that when X¹¹, X²², X³³ are —CH₂—; L¹ is —NHC(O)—; L² and L³ is —OC(O)—, R¹ is C₁₅ unsubstituted alkyl; R³ is methyl, ethyl, propyl, 2-methyl propyl, or cyclopropyl, then R² is not 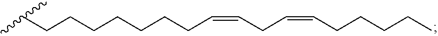 [SMILES: *CCCCCCC/C=C\C/C=C\CCCCC]
x) with proviso that when X¹¹, X²², X³³ are —CH₂—; each L¹, L² and L³ are —NHC(O)—; —O—, —SC(O), —OC(O)—, R¹ is C₁₅ unsubstituted alkyl; R³ is hydrogen or methyl, then R² is not 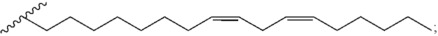 [SMILES: *CCCCCCC/C=C\C/C=C\CCCCC]
xi) with proviso that when X¹¹, X²², X³³ are —CH₂—; each L¹, L² and L³ are —NHC(O)—; —O—, —SC(O), —OC(O)—, one of R² and R³ is C₁₅ unsubstituted alkyl; and the other of R² and R³ is hydrogen, then R¹ is not 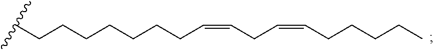 [SMILES: *CCCCCCC/C=C\C/C=C\CCCCC]
xii) with proviso that when X¹¹, X²², X³³ are —CH₂—; L¹ is —OC(O)—, R¹ is methyl or unsubstituted C₁₅ alkyl, L² is —O—, and R² is hydrogen, then L³-R³ is not OH or —OC(O)CH₃,
xiii) with proviso that when X¹¹ and X²² are —CH₂—; X³³ is —C(O)—; L¹ and L₂ are —OC(O)—; one of R¹ and R² is unsubstituted C₁₅ alkyl; the other of R¹ and R² is 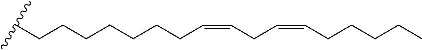 [SMILES: *CCCCCCC/C=C\C/C=C\CCCCC]
and L³ is —NH—; then R³ is not —CH₂—CH₃;
xiv) with proviso that when X¹¹ and X²² are —CH₂—; X³³ is —C(O)—; L¹ and L₂ are —OC(O)—; R² is unsubstituted C₁₅ alkyl; R¹ is 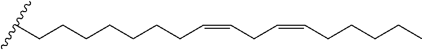 [SMILES: *CCCCCCC/C=C\C/C=C\CCCCC]
and L³ is —O—; then R³ is not —CH₂—CH₃.
xv) with proviso that when X¹¹, X²², X³³ are —CH₂—; L¹, L² and L³ are —OC(O)—; R¹ and R² are same as C₁₅ alkyl or 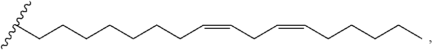 [SMILES: *CCCCCCC/C=C\C/C=C\CCCCC]
then R³ is not —CH₃,
xvi) with proviso that when X¹¹, X²², X³³ are —CH₂—; L¹, L² and L³ are —OC(O)—; R¹, R², R³ are same as unsubstituted C₇ or C₉ alkyl.

2. The compound of claim 1, wherein R^{2a} and R² together with atoms attached thereto are joined to form a substituted or unsubstituted C₅-C₅ cycloalkyl, substituted or unsubstituted 5 to 8 membered heterocycloalkyl.

3. The compound of claim 1 or 2, wherein the compound has the structure of: 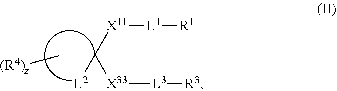
wherein L² is a bond, —C(O)—, —OC(O)—, —SC(O)—, —C(O)O—, —C(O)S—, —NHC(O)—, —C(O)NH—, —NHC(O)NH—, —NH—, —NCH₃—, —O—, —S—, —S(O)₂—, —NHS(O)₂—, —S(O)₂NH—, —NHC(O)O—, —OC(O)NH, substituted or unsubstituted C₁-C₃ alkylene, or substituted or unsubstituted 2 to 3 membered heteroalkylene;
z is an integer from 0 to 8;
R⁴ is halogen, —CX⁴₃, —CHX⁴₂, —CH₂X⁴, —OCX⁴₃, —OCH₂X⁴, —OCHX⁴₂, —N₃, —CN, —SO₂R^{4D}, —SO₂NR^{4A}R^{4B}, —NHC(O)NR^{4A}R^{4B}, —NO₂, —NR^{4A}R^{4B}, —C(O)R^{4C}, —C(O)—OR^{4C}, —C(O)NR^{4A}R^{4B}B, —OR^{4D}, —NR^{4A}SO₂R^{4D}, —NR^{4A}C(O)R^{4C}, —NR^{4A}C(O)OR^{4C}, —NR^{4A}OR^{4C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{4A}, R^{4B}, R^{4C}, and R^{4D} are independently hydrogen, —CX₃, —CHX₂, —CH₂X, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
X⁴ is independently —F, —Cl, —Br, or —I.

4. The compound of any one of claims 1 to 3, wherein the compound has the structure of: 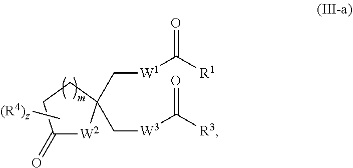
wherein each W¹, W², and W³ is independently —NH—, —O—, or —S—; and
m is an integer from 0 to 4.

5. The compound of claim 4, wherein the compound has the structure of:

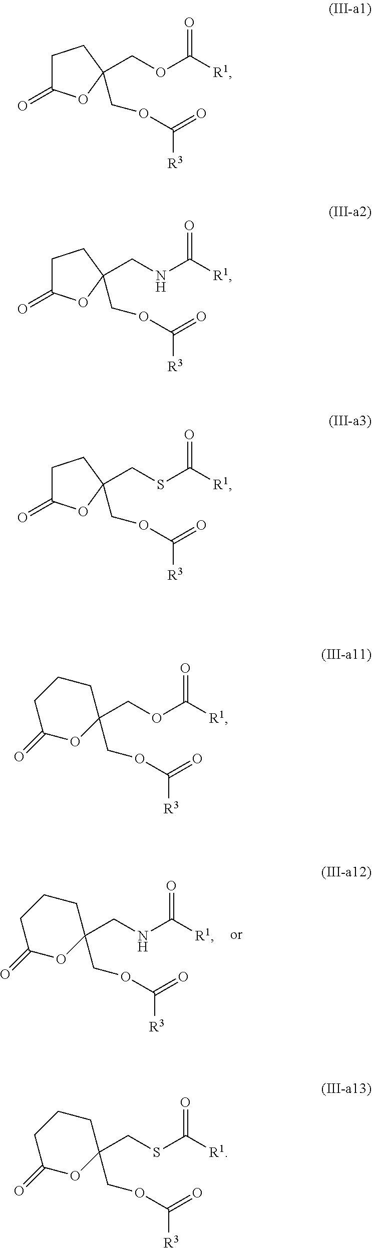

6. The compound of any one of claims 1 to 3, wherein the compound has the structure of: 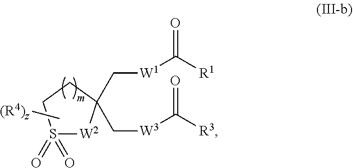
wherein each W¹, W², and W³ is independently —NH—, —O—, or —S—; and
m is an integer from 0 to 4.

7. The compound of claim 6, wherein the compound has the structure of:

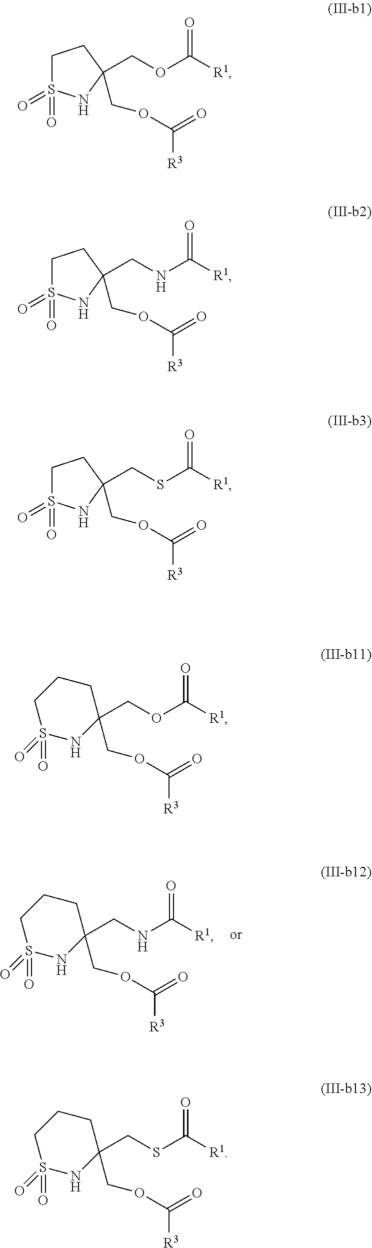

8. The compound of claim 1, wherein the compound has the structure of: 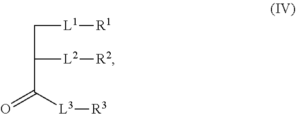
wherein each L¹, L², and L³ is independently a bond, —OC(O)—, —SC(O)—, or —NHC(O)—.

9. The compound of claim 8, wherein the compound has the structure of:

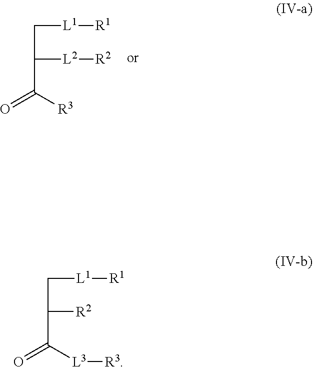

10. The compound of claim 1, wherein L² is —NR¹³C(O)—.

11. The compound of claim 10, wherein R² and R¹³ are joined to form a substituted or unsubstituted 5-8 membered heterocycloalkyl.

12. The compound of compound 11, wherein R² and R¹³ are joined to form

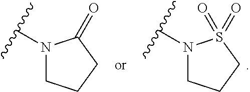 [SMILES: *N1CCCC1=O ; *N1CCCS1(=O)=O]

13. The compound of claim 1, wherein L³ is —NR¹⁵C(O)—.

14. The compound of claim 13, wherein R³ and R¹⁵ are joined to form a substituted or unsubstituted 5-8 membered heterocycloalkyl.

15. The compound of claim 14, wherein R³ and R¹⁵ are joined to form

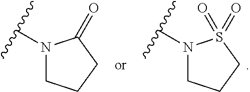 [SMILES: *N1CCCC1=O ; *N1CCCS1(=O)=O]

16. The compound of claim 1, wherein the compound has the structure of: 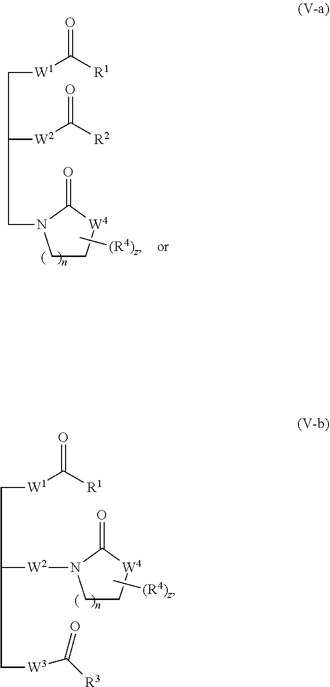
each W¹ and W² is independently a bond, —NH—, —O—, or —S—;
W⁴ is —NR¹⁶—, —CH₂—, —O—
n is an integer from 0 to 4;
z is an integer from 0 to 8;
R⁴ is halogen, —CX⁴₃, —CHX⁴₂, —CH₂X⁴, —OCX⁴₃, —OCH₂X⁴, —OCHX⁴₂, —N₃, —CN, —SO₂R^{4D}, —SO₂NR^{4A}R^{4B}, —NHC(O)NR^{4A}R^{4B}, —NO₂, —NR^{4A}R^{4B}, —C(O)R^{4C}, —C(O)—OR^{4C}, —C(O)NR^{4A}R^{4B}, —OR^{4D}, —NR^{4A}SO₂R^{4D}, —NR^{4A}C(O)R^{4C}, —NR^{4A}C(O)OR^{4C}, —NR^{4A}OR^{4C}, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{4A}, R^{4B}, R^{4C}, and R^{4D} are independently hydrogen, —CX₃, —CHX₂, —CH₂X, —COOH, —CONH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
X⁴ is independently —F, Cl, —Br, or —I.

17. The compound of claim 16, wherein the compound has the structure of:

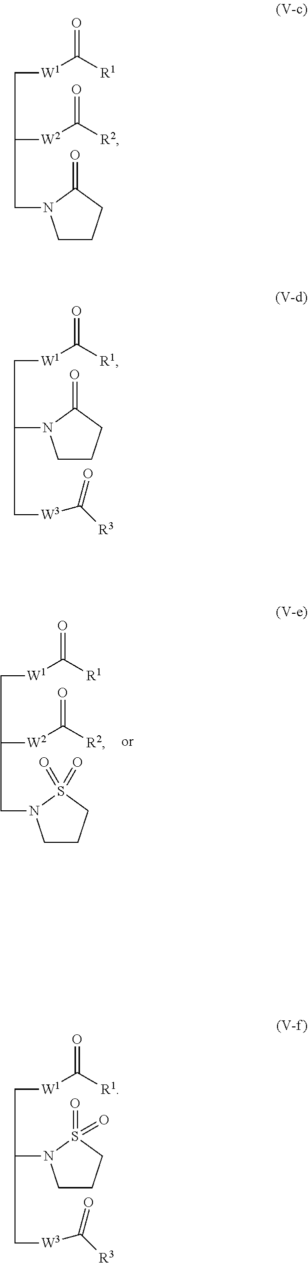

18. The compound of any one of claims 1 to 15, wherein:
R¹ is hydrogen, R^{1E}-substituted or unsubstituted C₁-C₂₀ alkyl, R^{1E}-substituted or unsubstituted 2 to 10 membered heteroalkyl, R^{1E}-substituted or unsubstituted C₃-C₈ cycloalkyl, or R^{1E}-substituted or unsubstituted phenyl, wherein R^{1E} is halogen, —OH, —NH₂, —COOH, —NO₂, —N₃, —CN, substituted or unsubstituted phenyl, a cholesterol or its derivative, a carbohydrate, —P(O)₂OH, —P(O)(OH)₂, a nucleic acid, or a peptide;
R² is hydrogen, R^{32E}-substituted or unsubstituted C₁-C₂ alkyl, R^{2E}-substituted or unsubstituted 2 to 10 membered heteroalkyl, R^{2E}-substituted or unsubstituted C₃-C₈ cycloalkyl, or R^{2E}-substituted or unsubstituted phenyl, wherein R^{2E} is halogen, —OH, —NH₂, —COOH, —NO₂, —N₃, —CN, substituted or unsubstituted phenyl, a cholesterol or its derivative, a carbohydrate, —P(O)₂OH, —P(O)(OH)₂, a nucleic acid, or a peptide; and
R³ is hydrogen, R^{3E}-substituted or unsubstituted C₁-C₂₀ alkyl, R^{3E}-substituted or unsubstituted 2 to 10 membered heteroalkyl, R^{3E}-substituted or unsubstituted C₃-C₅ cycloalkyl, or R^{3E}-substituted or unsubstituted phenyl, wherein R^{3E} is halogen, —OH, —NH₂, —COOH, —NO₂, —N₃, —CN, substituted or unsubstituted phenyl, a cholesterol or its derivative, a carbohydrate, —P(O)₂OH, —P(O)(OH)₂, a nucleic acid, or a peptide.

19. The compound of claim 18, wherein:
each R¹, R², and R³ is independently unsubstituted saturated C₁-C₂₀ alkyl.

20. The compound of claim 18, wherein:
each R¹, R², and R³ is independently unsubstituted saturated unbranched C₁-C₂₀ alkyl.

21. The compound of claim 18, wherein:
each R¹, R², and R³ is independently unsubstituted unsaturated C₁-C₂₀ alkyl.

22. The compound of claim 18, wherein:
each R¹, R², and R³ is independently unsubstituted unsaturated unbranched C₁-C₂₀ alkyl.

23. The compound of claim 18, wherein:
each R¹, R², and R³ is independently unsubstituted unsaturated C₁₀-C₂₀ alkyl.

24. The compound of claim 18, wherein:
each R¹, R², and R³ is independently unsubstituted unsaturated unbranched C₁₀-C₂₀ alkyl.

25. The compound of claim 18, wherein:
each R¹, R², and R³ is independently unsubstituted unsaturated unbranched C₁₀-C₂₀ alkyl.

26. The compound of claim 18, wherein:
each R¹, R², and R³ is independently unsubstituted saturated C₁-C₁₀ alkyl.

27. The compound of claim 18, wherein:
each R¹, R², and R³ is independently unsubstituted saturated C₁-C₄ alkyl.

28. The compound of claim 18, wherein:
one of R¹, R², and R³ is unsubstituted C₃-C₆ cycloalkyl.

29. The compound of claim 28, wherein:
one of R¹, R², and R³ is unsubstituted cyclopropyl or cyclohexyl.

30. The compound of claim 1, wherein the compound is:

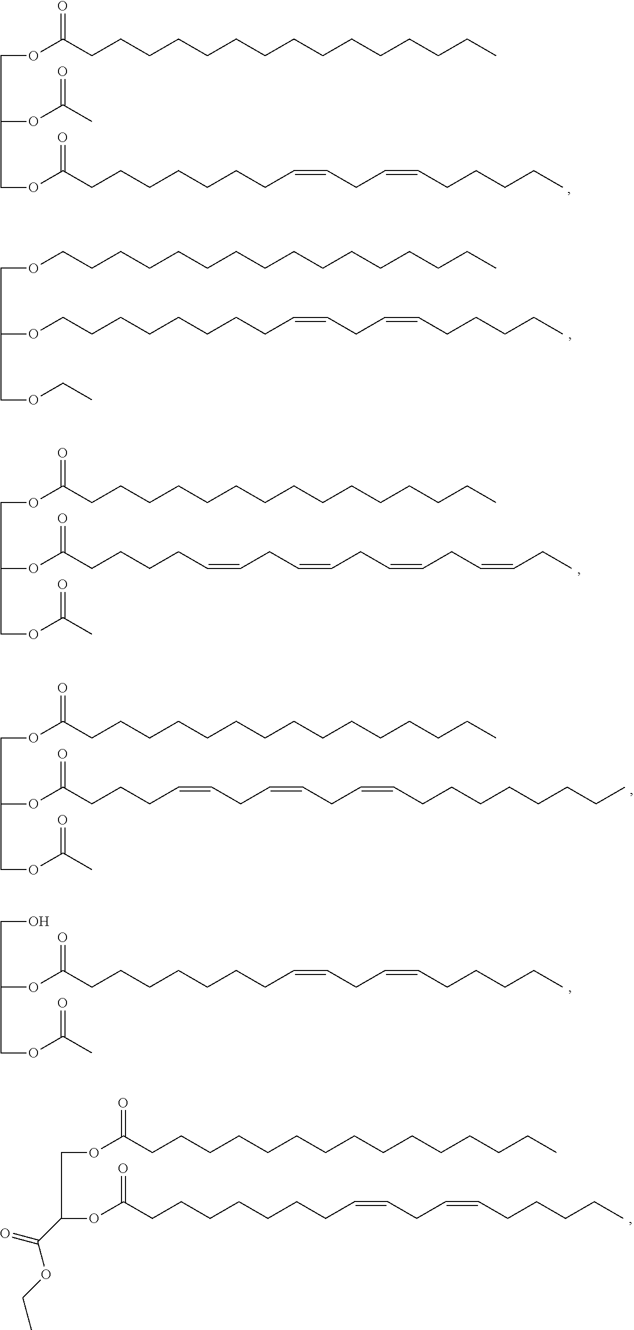 [SMILES: CCCCC/C=C\C/C=C\CCCCCCCC(=O)OCC(COC(=O)CCCCCCCCCCCCCCC)OC(C)=O ; CCCCC/C=C\C/C=C\CCCCCCCCOC(COCC)COCCCCCCCCCCCCCCCC ; CC/C=C\C/C=C\C/C=C\C/C=C\CCCCC(=O)OC(COC(C)=O)COC(=O)CCCCCCCCCCCCCCC ; CCCCCCCC/C=C\C/C=C\C/C=C\CCCC(=O)OC(COC(C)=O)COC(=O)CCCCCCCCCCCCCCC ; CCCCC/C=C\C/C=C\CCCCCCCC(=O)OC(CO)COC(C)=O ; CCCCC/C=C\C/C=C\CCCCCCCC(=O)OC(COC(=O)CCCCCCCCCCCCCCC)C(=O)OCC]

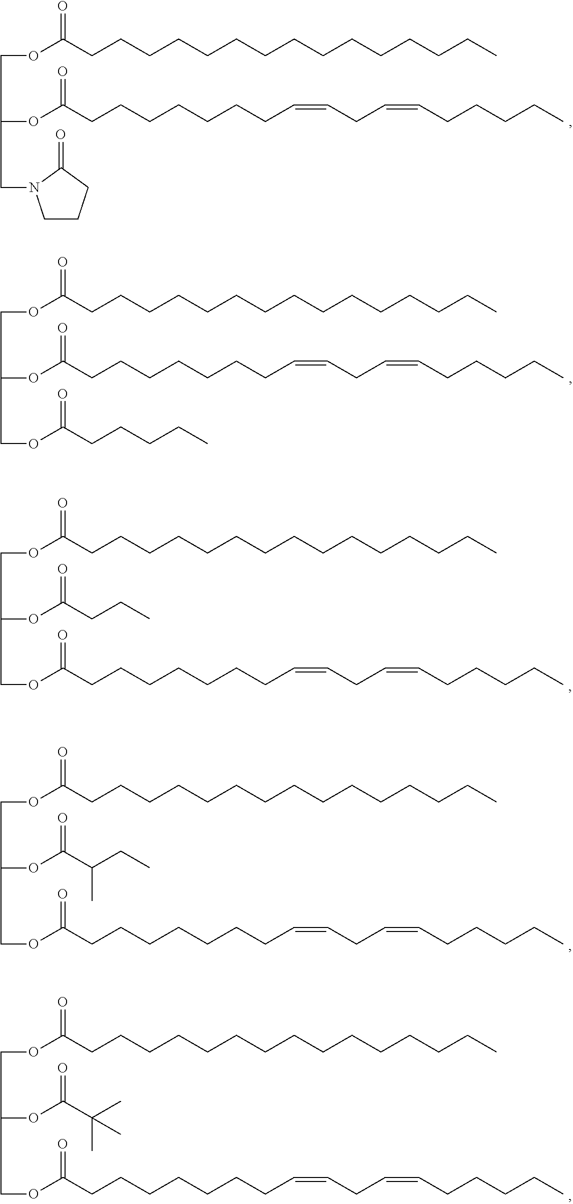

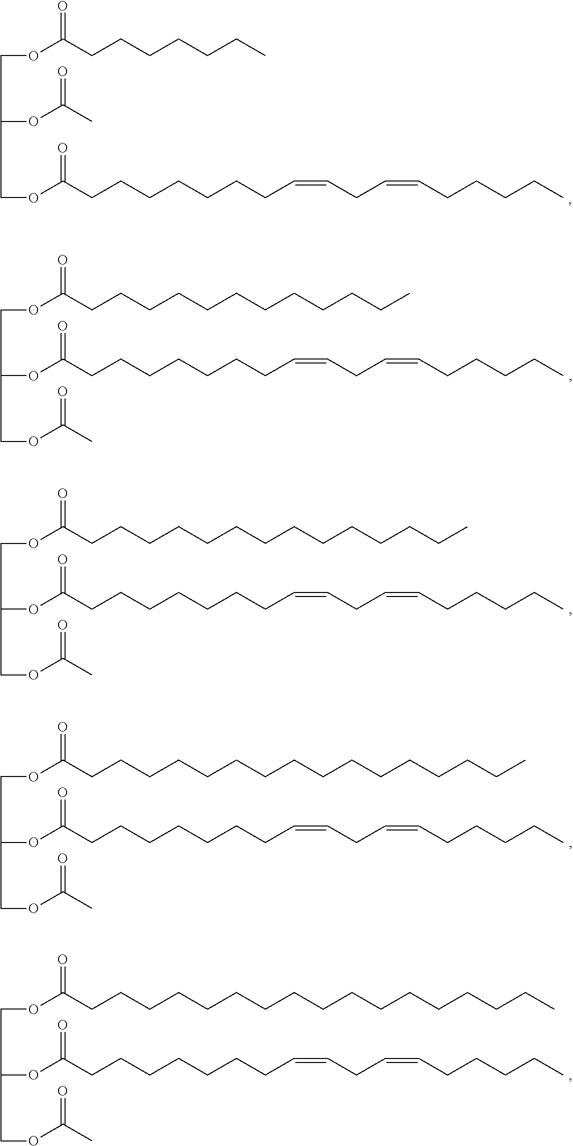 [SMILES: CCCCC/C=C\C/C=C\CCCCCCCC(=O)OCC(COC(=O)CCCCCCC)OC(C)=O ; CCCCC/C=C\C/C=C\CCCCCCCC(=O)OC(COC(C)=O)COC(=O)CCCCCCCCCCCC ; CCCCC/C=C\C/C=C\CCCCCCCC(=O)OC(COC(C)=O)COC(=O)CCCCCCCCCCCCCC ; CCCCC/C=C\C/C=C\CCCCCCCC(=O)OC(COC(C)=O)COC(=O)CCCCCCCCCCCCCCCC ; CCCCC/C=C\C/C=C\CCCCCCCC(=O)OC(COC(C)=O)COC(=O)CCCCCCCCCCCCCCCCC]

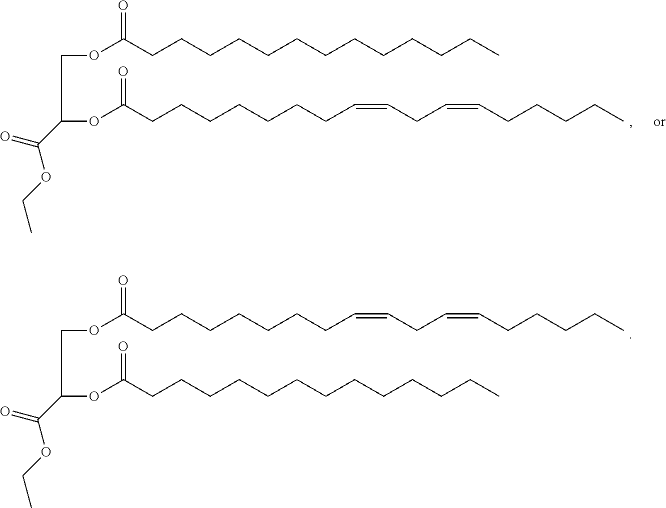 [SMILES: CCCCC/C=C\C/C=C\CCCCCCCC(=O)OC(COC(=O)CCCCCCCCCCCCC)C(=O)OCC ; CCCCC/C=C\C/C=C\CCCCCCCC(=O)OCC(OC(=O)CCCCCCCCCCCCC)C(=O)OCC]

31. A pharmaceutical composition comprising a compound of any one of claims 1 to 30, and a pharmaceutically acceptable excipient.

32. A method of treating exposure to ionizing radiation in a subject comprising administering a compound of any one of claims 1 to 30 or a pharmaceutical composition of claim 31 to a subject in need thereof.

33. The method of claim 32 wherein the subject has been identified as suffering from exposure to ionizing radiation and the compound is administered to the identified subject.

34. A method of treating acute lung injury in a subject comprising administering a compound of any one of claims 1 to 30 or a pharmaceutical composition of claim 31 to a subject in need thereof.

35. The method of claim 34 wherein the subject has been identified as suffering from acute lung injury and the compound is administered to the identified subject.

36. A method of treating mucositis in a subject comprising administering a compound of any one of claims 1 to 30 or a pharmaceutical composition of claim 31 to a subject in need thereof.

37. The method of claim 36 wherein the subject has been identified as suffering from mucositis radiation and the compound is administered to the identified subject.

38. A method of treating a subject suffering from cancer comprising administering a compound of any one of claims 1 to 30 or a pharmaceutical composition of claim 31 to a subject in need thereof.

39. The method of claim 38 wherein the subject has been identified as suffering from cancer and the compound is administered to the identified subject.

40. A method of any one of claims 32 through 39 wherein the subject is a human.

41. A kit for treating exposure to ionizing radiation comprising a compound of any one of claims 1 through 30 or a pharmaceutical composition of claim 31.

42. The kit of claim 41 further comprising instructions for treating exposure to ionizing radiation.

43. A kit for treating acute lung injury comprising a compound of any one of claims 1 through 30 or a pharmaceutical composition of claim 31.

44. The kit of claim 43 further comprising instructions for treating acute lung injury.

45. A kit for treating mucositis comprising a compound of any one of claims 1 through 30 or a pharmaceutical composition of claim 31.

46. The kit of claim 45 further comprising instructions for treating mucositis.

47. A kit for treating mucositis comprising a compound of any one of claims 1 through 30 or a pharmaceutical composition of claim 31.

48. The kit of claim 47 further comprising instructions for treating cancer.
